(19) United States
(12) Patent Application Publication
(10) Pub. No.: US 2005/0227231 A1
(43) Pub. Date: Oct. 13, 2005

(54) Device for sequencing nucleic acid molecules

(76) Inventor: Dimitri Tcherkassov, Lubeck (DE)

Correspondence Address: MCDERMOTT, WILL & EMERY, 4370 LA JOLLA VILLAGE DRIVE, SUITE 700, SAN DIEGO, CA, 92122, US

(21) Appl. No.: 10/491,557

(22) PCT Filed: Oct. 2, 2002
(86) PCT No.: PCT/EP02/11098

§ 371 Date: Feb. 15, 2005

(30) Foreign Application Priority Data
Oct. 4, 2001 (DE) 101 48 868.8

(51) Int. Cl. C12Q 1/68; C12M 1/34

(52) U.S. Cl. 435/6; 435/287.2

(57) ABSTRACT

The invention relates to a device for the automatic determination of nucleic acid sequences. The sequencing reaction occurs by the parallel sequential construction of the strands complementary to individually-fixed single-strand nucleic acid chains. The automatic sequencing carries out said sequential construction and detects electromagnetic radiation from individual marked nucleotides (NT*s) incorporated in the complementary strands. The sequence of the immobilised nucleic acid chain is determined from the order of the incorporated NT*s.

### INTRODUCTION

The subject matter of the invention is a device for the automatic determination of nucleic acid sequences. The sequencing reaction occurs by the parallel sequential construction of the strands complementary to individual fixed single-strand nucleic acid chains. The automatic sequencing device carries out said sequential construction and detects electromagnetic radiation from individual labelled nucleotides (NT*s) incorporated into the complementary strands. The sequence of the immobilised nucleic acid chains is determined from the order of the incorporated NT*s.

### 1. ABBREVIATIONS AND EXPLANATIONS OF TERMS

DNA—Deoxyribonucleic acid of different origins and different lengths (genomic DNA, cDNA, ssDNA, dsDNA)

RNA—Ribonucleic acid (usually mRNA).

Polymerases—Enzymes which are capable of incorporating complementary nucleotides into a growing DNA or RNA strand (e.g. DNA polymerases, reverse transcriptases, RNA polymerases).

dNTP—2′-Deoxynucleoside triphosphates as substrates for DNA polymerases and reverse transcriptases.

NT—natural nucleotide, usually dNTP, unless expressly characterised differently.

The abbreviation “NT” is also used to indicate the length of a nucleic acid sequence, e.g. 1,000 NT. In this case, “NT” stands for nucleoside monophosphate.

In the text, the plural of abbreviations is formed by using the suffix “s”, “NT”, for example, stands for “nucleotide”, “NTs” stands for several nucleotides.

NT*—a nucleotide reversibly modified with a fluorescent dye and a group leading to termination, usually dNTP, unless expressly characterised differently. NTs* means: modified nucleotides.

NAC—stands for a nucleic acid chain (DNA or RNA). NACs means several different or identical nucleic acid chains. NACs include e.g. single-strand or double-strand oligonucleotides or polynucleotides, genomic DNA, populations of cDNAs or mRNAs.

NACF—nucleic acid chain fragment, NACFs—nucleic acid chain fragments. Fragments of NACs (DNA or RNA) which are formed after a fragmenting step. The automated sequencing device can be used both for the analysis of NACs and NACFs. An essential difference between NACs and NACFs consists of the preparation of the material and the analysis of the sequences obtained. There are no major differences in the sequencing reaction and the course of the process steps such that many process steps are described jointly for NACs and NACFs.

Plane surface—surface which preferably exhibits the following characteristics: 1) It allows several individual molecules, preferably more than 100, even more preferably more than a 1000 to be detected simultaneously with the lens system to surface distance given in each case at a given position of the lens system. 2) The immobilised individual molecules are present in the same focus plane which can be adjusted reproducibly.

Definition of termination: in this patent application, termination means the reversible stop of the incorporation of the modified NTs*. The modified NT*s carry a reversibly coupled group leading to termination. This group can be removed from the incorporated NT*s.

This term must not be confused with the usual meaning of the word “termination” by dideoxy-NTP in conventional sequencing.

Gene products—mRNA transcripts or nucleic acid chains derived from mRNA (e.g. single-strand cDNA, double-strand cDNA synthesised from single-strand cDNA, RNA derived from cDNA or DNA amplified from cDNA). Gene products can also be referred to as gene sequence equivalents.

SNP—Single nucleotide polymorphism

PBS—Primer binding site

Object field—part of the reaction surface the image of which can be taken by the camera with a defined X, Y setting of the lens system.

Sequencing reaction—the sum of individual process steps up to the result: the sequences determined of individual NACs immobilised on the solid surface.

DPuMA—German Patent and Trademark Office

### 2. STATE OF THE ART

The technique most frequently used to analyse nucleic acid sequences is dideoxy sequencing according to Sanger. In this case, labelled nucleic acid chain fragments are separated in a gel according to their length. One example of such an automatic sequencing device is described in EP 0 294 524. This automatic sequencing device is capable of analysing up to 100 sequences simultaneously.

In the present invention, a sequencing device is presented which is capable of analysing more than 100,000 nucleic acid sequences in parallel and thus exhibits substantially greater sequencing velocities in comparison with a “state of the art” sequencing device. This automatic sequencing device allows both the qualitative analysis of sequences (sequencing in the narrow sense of the word) and quantitative analysis (evaluation of the number of sequences determined, e.g. by gene expression analysis).

Such an automatic sequencing device can be used in many areas, e.g. in medicine, the pharmaceutical area and biotechnology.

### 3. GENERAL DESCRIPTION

An essential subject matter of this invention is a device, an automatic sequencing device, for the automatic parallel identification of nucleic acid sequences. The automatic sequencing device according to the invention is capable of sequencing several hundred thousand individual immobilised nucleic acid chains in parallel. A de novo sequencing of nucleic acid chains, an analysis of sequence variants or a gene expression analysis are possible by means of the automatic sequencing device described. Consequently, this automatic sequencing device represents a universal automatic device for the analysis of nucleic acid sequences.

Important parts of the automatic sequencing device according to the invention are:
a housing
an optical system with a source of light, a filter device
a detection device
a reaction platform
a translation system (scanning table)
and a computer system for the control of individual steps of the sequencing process and signal analysis.

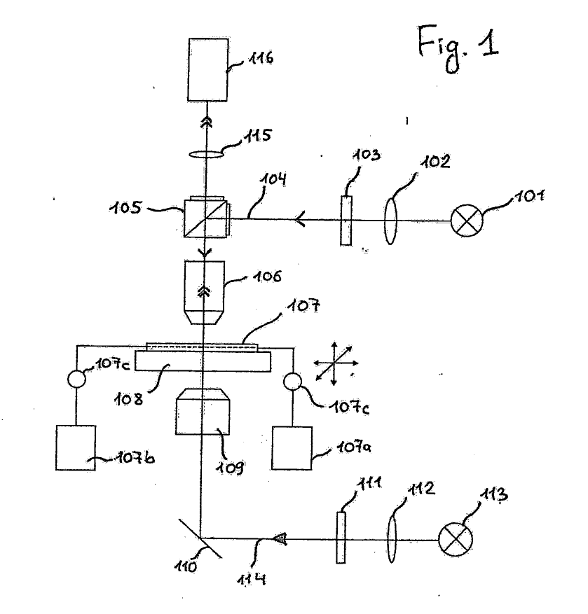

A diagrammatic example of the automatic sequencing device is illustrated in FIG. 1.

The sequencing reaction takes place by the sequential construction of the strands complementary to the individual fixed single-strand nucleic acid chains. The automatic sequencing device carries out this sequential construction and detects electromagnetic radiation (fluorescence signals) from individual labelled nucleotides (NT*s) incorporated into the complementary strands.

Examples of such a sequencing reaction are described in the patent applications of Tcherkassov et al (“Verfahren zur Bestimmung der Genexpression” (Process for the determination of gene expression) DPuMA file number 101 20 798.0-41, “Verfahren zur Analyse von Nukleinsäureketten” (Process for the analysis of nucleic acid chains) DPuMA file number 101 20 797.2-41, “Verfahren zur Analyse von Nukleinsäurekettensequenzen und der Genexpression” (Process for the analysis of nucleic acid chain sequences and gene expression) DPuMA file number 101 42 256.3). The sequencing reaction includes essentially the following steps:

1) The preparation for cyclic steps consisting of:
a) Sample preparation in the case of which single-strand nucleic acid chains (NACs) between 20 and 5,000 in length, preferably between 50 and 1,000 NT in length, are made available and, if necessary, provided with a PBS; in the case of longer sequences, a fragmentation step is carried out such that NACFs are formed.
b) A step of fixing the prepared NAC sample to the reaction surface in the form of NAC primer complexes or NACF primer complexes. In this case, the individual NACs or NACFs are fixed to the reaction surface in such a way that an enzymatic reaction (synthesis of the complementary strand) can take place at these molecules, compare Example of Immobilisation.

2) After fixing of the NACs or NACFs in the form of NAC primer complexes or NACF primer complexes, the cyclic steps are commenced with all the complexes immobilised on the surface. The synthesis of the complementary strand to each individual fixed NAC or NACF serves as the basis for sequencing. Labelled NTs* are incorporated into the newly synthesised strand during this process. These NT*s are modified in such a way that the polymerase is only capable of incorporating one single labelled NT* into the growing chain in one cycle. This modification of the NT*s is reversible such that, after removal of this modification, a further synthesis can take place. The sequencing reaction takes place in several cycles. One cycle comprises the following steps (cyclic steps):
a) Addition of a solution with labelled nucleotides (NTs*) and polymerase to immobilised nucleic acid chains,
b) Incubation of the immobilised nucleic acid chains with this solution under conditions suitable for extending the complementary strands by one NT,
c) Washing,
d) Detection of the signals from individual incorporated NT*s,
e) Removal of the fluorescent label and the group leading to termination from the incorporated nucleotides,
f) Washing.

3) From the sequence of the detected signals of the incorporated NT*s, the specific sequence is determined for each immobilised NAC and/or NACF participating in the reaction.

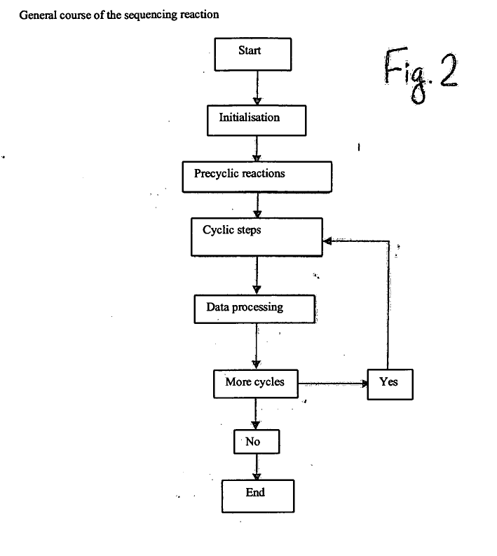

An example of the general course of the sequencing reaction is illustrated in FIG. 2.

The NT*s that can be used in the process are reversibly labelled with a dye. The criteria for selecting these dyes are indicated in the example (Dye). This dye is coupled to the nucleotide and can be cleaved off by chemical or photochemical reaction. For example, the NT*s detailed in the patent applications of Tcherkassov et al (“Verfahren zur Bestimmung der Genexpression” (Process for the determination of gene expression) DPuMA file number 101 20 798.0-41, “Verfahren zur Analyse von Nukleinsäureketten” (Process for the analysis of nucleic acid chains) DPuMA file number 101 20 797.2-41, “Verfahren zur Analyse von Nukleinsäurekettensequenzen und der Genexpression” (Process for the analysis of nucleic acid chain sequences and gene expression) DPuMA file number 101 42 256.3) can be used. The detailed information regarding the process, synthesis and application of NT*s, including the selection of polymerase, the reaction conditions for the incorporation of NT* and cleavage are illustrated in the above-mentioned sources.

The reaction conditions of step (b) in one cycle are selected such that the polymerases are capable of incorporating a labelled NT* into more than 50%, preferably more than 90%, of the NAC's and/or NACFs participating in the sequencing reaction, in one cycle.

The number of cycles to be carried out depends in this respect on the task in hand, is theoretically not limited and is preferably between 20 and 5,000.

A further subject matter of this invention consists of a reaction platform for the execution of chemical and biochemical reactions with individual molecules, in particular for the execution of sequential reactions with individual nucleic acid chains immobilised on the surface. This reaction platform is preferably part of the automated sequencing device according to the invention.

The use of the automated sequencing device is illustrated by way of two embodiments.

In one embodiment, the automatic sequencing device is used to sequence long (more than 100 kb) nucleic acid chains.

In this case, a population of relatively small, overlapping, single-strand nucleic acid chain fragments (NACFs) is generated from one long nucleic acid chain (NAC), these fragments are provided with a primer suitable for the start of the sequencing reaction, fixed in the reaction platform and sequenced.

From the overlapping NACF sequences, the original NAC sequence can be reconstructed (“Automated DNA sequencing and analysis” page 231 ff. 1994 M Adams et al. Academic Press, Huang et at. Genom Res. 1999 volume 9, page 868, Huang Genomics 1996 volume 33, page 21, Bonfield et al. NAR 1995 volume 23, page 4992, Miller et al. J. Comput. Biol 1994 volume 1, page 257). In this process, the entire population of NACF sequences is examined for agreements/overlaps in the NACFs sequences. By means of these agreements/overlaps, the NACFs can be combined and a larger coherent sequence reconstructed e.g.

```
CGTCCGTATGATGGTCATTCCATG
                              CATTCCATGGTACGTTAGCTCCTAG
                                                                    TCCTAGTAAAATCGTACC:
```

In practice, it has proved advantageous during sequencing of unknown sequences, to achieve a length of more than 300 bp of the sequenced sections. This allows sequencing of genomes of eukaryotes by the shotgun method.

In another embodiment, the automated sequencing device is used for gene expression analysis. This method is based on several principles:

1) Short nucleotide sequences (10-50 NTs) contain sufficient information for identifying the corresponding gene if the gene sequence itself is already contained in a data bank.

A sequence of, for example, 10 NTs can form more than 106 different combinations. This is, for example, sufficient for most genes in the human genome which, according to present day estimates, contains 32,000 genes. For organisms with fewer genes, the sequence can be shorter.

2) The method is based on sequencing of individual nucleic acid chain molecules.

3) Nucleic acid chain mixtures can be examined.

4) The sequencing reaction takes place simultaneously on many molecules, the sequence of each individual immobilised nucleic acid chain being analysed.

It is well known that, for the investigation of gene expression, mRNAs or nucleic acid chains derived from mRNA (e.g. single-strand cDNAs, double-strand cDNAs, RNA derived from cDNA or DNA amplified from cDNA) can be used. Irrespective of the exact composition, they will be referred to as gene products in the following. Partial sequences of these gene products, too, will be referred to as gene products in the following.

These gene products represent a mixture of different nucleic acid chains.

The gene products are converted into the single-strand form, provided with a primer, fixed on the reaction surface and sequenced.

The sequences of the immobilised gene products determined are compared with each other to determine the abundances and allocated to certain genes by comparison with gene sequences in databanks.

### 3a. DETAILED DESCRIPTION OF PREFERRED EMBODIMENTS OF THE AUTOMATED SEQUENCING DEVICE

Detection Device

For the detection of the fluorescence of individual molecules, near field microscopy (NFM), laser scanning microscopy, total internal reflection microscopy (TIRM) and epifluorescence microscopy, for example, are used. These techniques differ by their physical principles and the design of their optical systems (Science 1999 volume 283 1667, Unger et al. BioTechniques 1999 volume 27 page 1008, Ishijaima et al. Cell 1998 volume 92 page 161, Dickson et al. Science 1996 volume 274 page 966, Xi et al. Science 1994 volume 265 page 361, Nie et al. Science 1994 volume 266 page 1018, Betzig et al. Science 1993 volume 262 page 1422). The automated sequencing device according to the invention employs the epifluorescence mode as principle of microscopy. This mode is preferably used because it differs from TIRM, laser scanning microscopy and NFM by having several advantages such as:

1) the size of the 2D image formed by taking a picture e.g. with a CCD camera and which can contain more than 1000 signals of individual molecules (it is, for example, possible to prepare an image of more than 100 μm×100 μm with a 100× NA 1.4 lens system.

2) The excitation and fluorescent light are passed through the same optical system to the object under investigation. As a result, only the surface of the object contributing to the formation of the image is exposed, the neighbouring regions are not exposed.

3) Such a system is cost-advantageous in comparison with a laser scanning system.

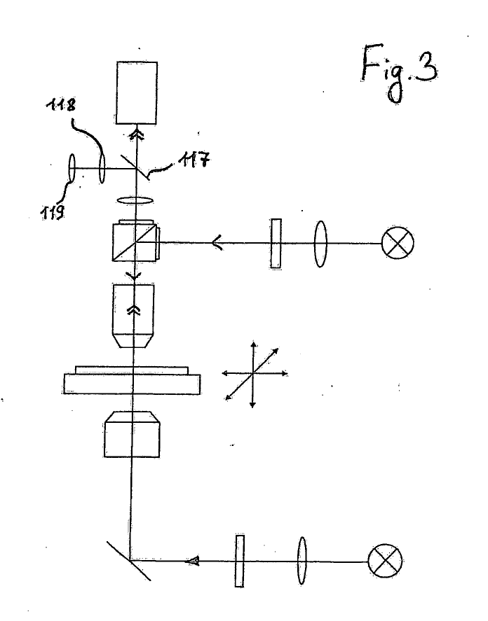

The housing, the translation device (scanning table), the optical system with magnification device (lens system), the sets of filters, the dichroic mirror (a colour separator), the source of light and other auxiliary devices such as the light source cooler, light reflector, apertures etc are commercially available as “state of the art” wide field epifluorescence microscope (companies: Zeiss, Nikon, Olympus). The schematic design of a “state of the art” epifluorescence microscope is illustrated in FIG. 3. Such a microscope can be integrated into the automated sequencing device. The following microscopes are examples: Axioskop (Zeiss), Axioplan 2 (Zeiss), Axiovert 100TV, 135TV, 200 (Zeiss), Olympus IX 70 (Olympus), Olympus BX 61 (Olympus), Eclipse TE 300 (Nikon), Eclipse E800 (Nikon). These microscopes are used by the persons skilled in the art as examples of individual components of the automated sequencing device. In the automated sequencing advice according to the invention, other equivalent functional units can also be used.

In the following, the essential parts of the equipment will be described as an example.

An upright or an inverted microscope can be used. To simplify the illustration rather than as a restriction, an upright microscope is illustrated in the following (In both cases it is preferred to use epifluorescence illuminations; essentially, this means that the excitation light and the fluorescent light are passed through the same optical system of the lens system).

Different sources of light can be used. The source of light can be integrated into the automated sequencing device or coupled to it via a light conductor.

Sources of light with continuous or line-shaped spectra can be used. The spectral properties of the source of light must correspond to the requirements of fluorescence excitation of the fluorescent dyes, compare Example of Dyes. Both visible and infrared light can be used for excitation purposes, it being possible for a source of light to be used for one and for several dyes.

The intensity of the excitation light at defined wavelengths is between 10 W/cm² and 100,000 W/cm², preferably between 100 W/cm² and 100,000 W/cm² on the illuminated reaction surface (10,000 W/cm²=10 mW/100 μm²). In a preferred embodiment, a lamp is used as the source of light. For example, Xe, Hg/Xe or “metal halide” arc lamps can be used, e.g. a mercury vapour short arc lamp HBO 50, HBO 100 or HBO 200. The use of a lamp is preferable to a laser because:
1) the object field whose 2D image (e.g. 100 μm×100 μm) is taken is illuminated almost homogeneously,
2) Light with different wavelengths is produced such that a lamp can be used to stimulate the fluorescence of several dyes,
3) Lamps are capable of producing UV light more cost effectively compared with lasers.

In another preferred embodiment, one or several lasers are used (e.g. a Nd:YAG laser, Antares, Coherent with a double frequency, 532 nm, to stimulate Cy3 dye and an Nd:YAG-pumped dye laser, Coherent 700, to stimulate Cy5 dye at 630 nm). The advantage of a laser consists of its longer service life and its great intensity of the excitation light.

Laser diodes can also be used as source of light.

The exposure time is preferably between 0.1 milliseconds (ms) and 20 seconds (s), more preferably between 1 ms and 1 s. It is controlled by an acoustooptical or electrooptical modulator, for example, or by a shutter which is controlled by the principal computer. The shutter may consist of a mechanical slide.

For the selection of excitation and fluorescent light and to reduce the scattered light, filters are preferably used. They are, for example, commercially available (Zeiss, Nikon, Olympus, Leica) and need to be adjusted to the corresponding dyes used for the sequencing reaction. Usually, several filters are combined to form a set of filters. Such a set of filters usually consist of a filter for the selection of the excitation light, a colour separator (dichroic mirror) and a filter for the selection of the fluorescent light. Commercially, both mono-band filter combinations (for one dye e.g. Cy3 or Cy5) and multi-band filter combinations (for several dyes, e.g. Cy3-CY5 combination) are available (e.g. from Zeiss, Nikon, Leica, Olympus).

Preferably, the filters are fixed in a mount. This mount allows individual filters or sets of filters to be replaced. Both a filter revolver and a filter slide are known as being state of the art. The replacement of the sets of filters, for example, takes place automatically by means of a filter revolver driven by a motor and controlled by the principal computer.

The excitation and fluorescent light is passed through a lens system. Preferably, PlanNeofluar and PlanApochromat lens systems, preferably oil immersion lens systems with a 40 to 100 fold magnification and with an NA of preferably more than 1.2 are used such as PlanNeofluar 100×, NA 1.4 (Zeiss), PlanApochromat 100× NA 1.4 (Zeiss), PlanApo 100× NA 1.4 Olympus Japan. Preferably, immersion oil with a low inherent fluorescence is used, e.g. from Cargille Laboratories, Cedar Grove, N.J., USA. Glycerine or water can also be used as immersion medium with corresponding immersion lens systems.

The fluorescent light of the incorporated nucleotides is collected with a lens system (O) and passed to the detection device (D). This detection device preferably consists of a cooled CCD camera or an intensified CCD camera (K). Many variations of cameras are commercially available such as SenSys™ (from Photometrix), AxioCam (from Zeiss) or 1-PentaMAX (from Roper Scientific, Trenton, N.J., USA).

Preferably, CCD chips with a high resolution are used. This allows, on the one hand, a better identification of the signals of individual molecules, and/or a better differentiation between closely situated signals (compare Example of Detection), on the other hand, each time an image is taken, this is taken of a large object surface and consequently a large number of signals are simultaneously recorded in the case of a sufficient specificity of signal recognition.

Modern cameras allow such image taking and have CCD chips with a resolution of preferably at least 512×512 pixel, ideally more than 1000×1300 pixel and a pixel size of approximately 51 μm×5 μm.

Both a black and white camera (SW camera) and a colour camera can be used. For the SW camera, fluorescent light from the same dyes is selected with a mono-band filter combination. In the case of a colour camera, multi-band filter combinations can be used.

Using the camera, a 2D image is produced which reflects signal intensities as a function of x, y co-ordinates. This image is analysed by an image processing program which differentiates both between the signals of incorporated NT*s and the background signal as well as being capable of differentiating between signals lying close together. An example of the operating principle of such a program is described in the example “Detection”.

Preferably, a controlled scaning table is used as translation device. Such tables are commercially available (Märzhäuser Wetzlar, Zeiss, Leica, Olympus and Nikon). The control is effected by a motor which is controlled by the principal computer. These tables must be capable of adjusting the same X-Y-Z co-ordinates over several cycles. Preferably, the deviation from a defined position (x-y-z)i is less than 5 μm, ideally less than 0.1 μm during the entire sequencing reaction.

The principal computer (C) is connected to the detection device and the reaction platform and controls the course of the sequencing reaction. As comprehensive an automation of the operation of the automated sequencing device as possible is aimed for. In this connection, the following events and/or parts in the automated sequencing device are preferably automated:
1) all events in the solution exchange at the reaction surface
2) all events during detection
3) all signal processing steps up to the sequence composition.

In one embodiment, the principal computer has also access to genetic databanks and is able to carry out sequence composition and sequence recognition.

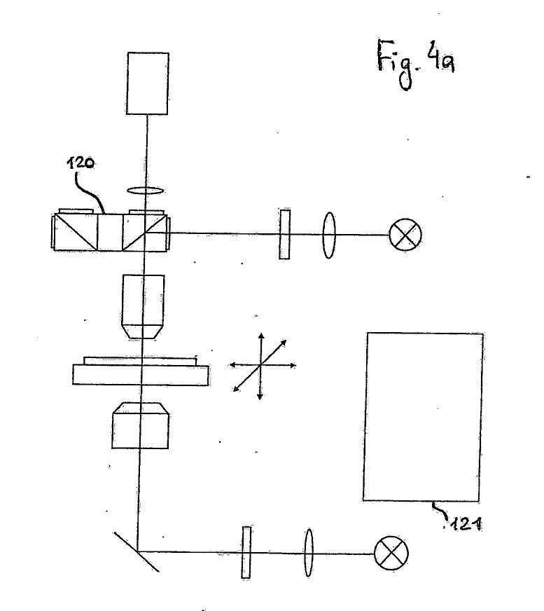

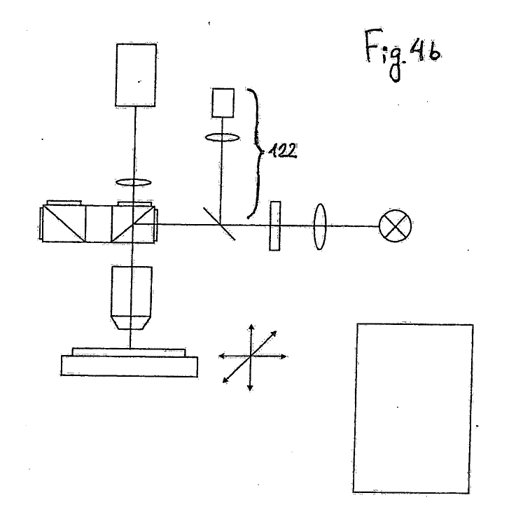

FIG. 4a and FIG. 4b illustrate exemplary embodiments of the detection device of the automated sequencing device according to the invention, a lamp serving as source of light.

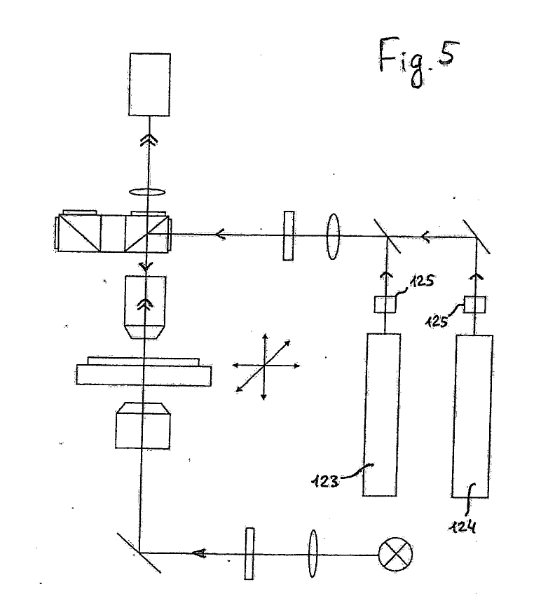

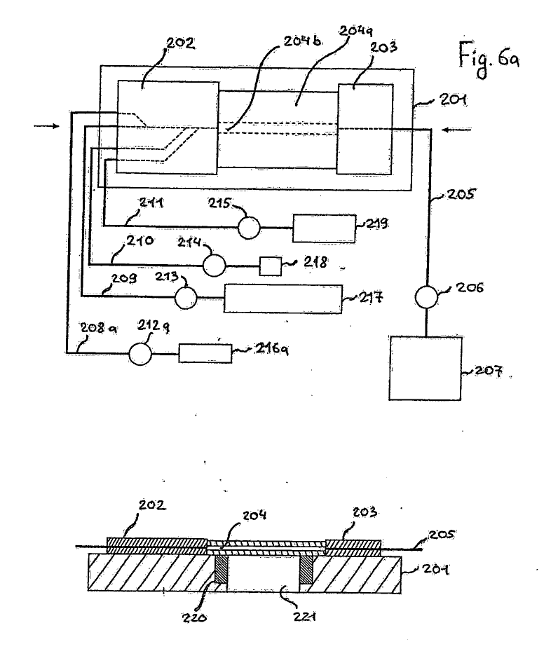

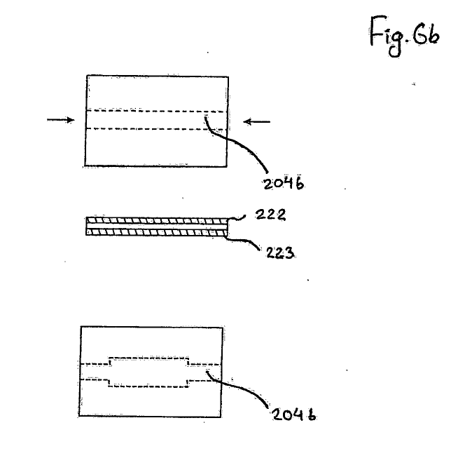

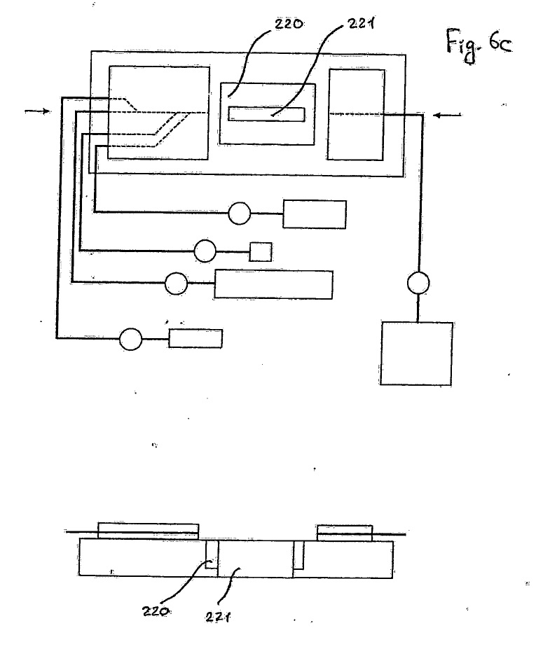

In FIG. 5, an exemplary arrangement of a detection device with 2 lasers is illustrated.

Reaction Platform

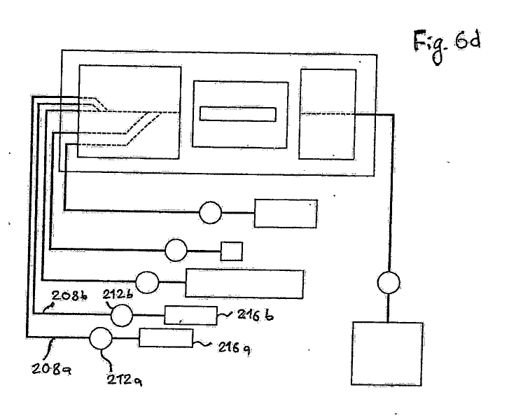

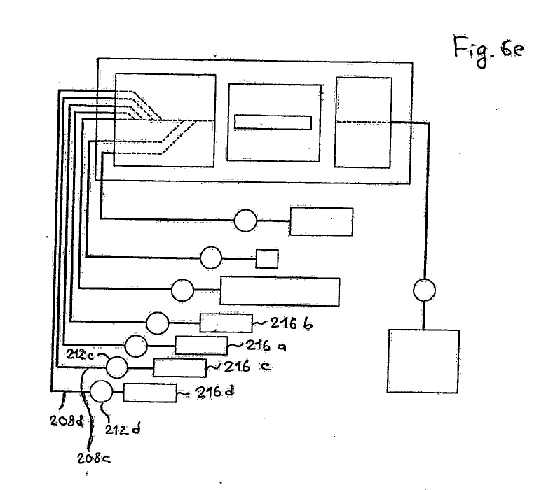

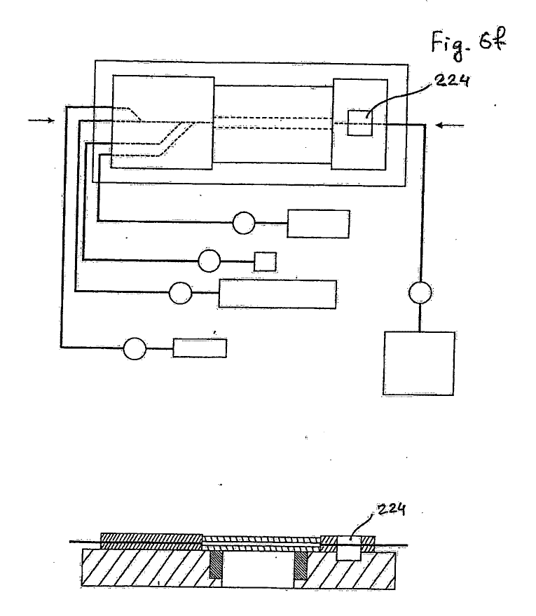

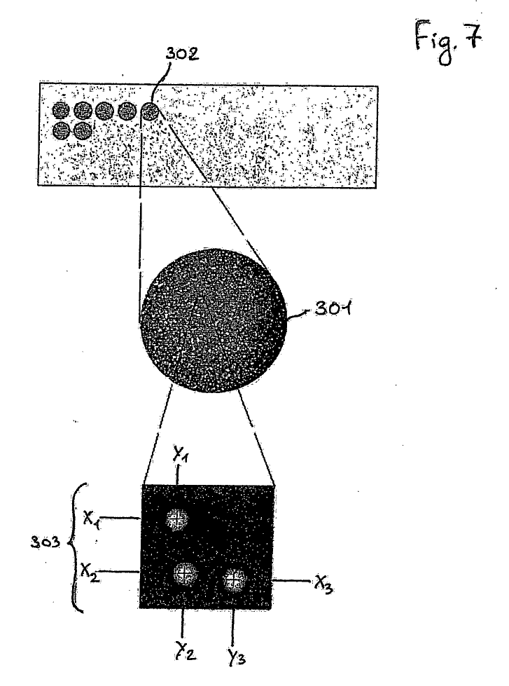

The reaction platform preferably represents a controlled through-flow device. It is equipped with one or several reaction surfaces and allows a controlled sequential exchange of reaction solutions such that the execution of sequential reactions is possible on these surfaces. In the following, an embodiment of such a reaction platform is to be illustrated as an example (FIG. 6a).

One or several reaction platforms can be used simultaneously. A parallel arrangement of two reaction platforms allows scanning of reaction platform (1) while the biochemical reactions take place in the reaction platform (2). The reaction platforms are fixed to the scanning table and moved by the latter.

In a preferred embodiment, the reaction platform consists of 3 parts (FIG. 6a):
1) the replaceable part, a chip (204a) with a microfluid channel (204b), MFC, which carries the reaction surface and is preferably used for only one sequencing analysis;
2) a stationary part, the distribution device (distributor) (FIG. 6c) which controls the replacement of solutions in the MFC; here, the MFC is connected to the distributor in such a way that solutions can be automatically supplied to the MFC and removed from it.
3) a further stationary part of the reaction platform, a thermostat unit (FIG. 6c, 220) by means of which the temperature in the MFC can be controlled (thermoblock).

An example of the construction of a chip with the MFC is illustrated diagrammatically in FIG. 6b. It consists of two plates (222, 223) and 2 spacers such that a channel (204b) is formed between the two plates. The height of this channel is preferably between 5 and 200 μm, its width between 0.1 and 10 mm and its length between 10 and 40 mm. The cover plate of the MFC facing the lens system is equipped with a surface permeable to the excitation and fluorescent light, or a window, preferably of glass. The chip itself can be constructed e.g. of glass or plastic (e.g. PMMA, PVC, polycarbonate).

In another embodiment, the chip has several MFCs (e. 2 or 3 or 4), the replacement of solution in these MFCs can take place by being independently controlled from each other by the distributor. In this way, different cycle steps can take place in parallel in one chip such that the analysis time is reduced.

In the following, a chip with only one MFC is considered as an example.

The replacement of liquids in the MFC is controlled by the distributor (FIG. 6a, c, d, e, f). In one embodiment, it consists of a structural element with integrated controlled valves, feed hoses and one or several pumps. Their number and exact arrangement must be adjusted to each design. The liquid transport in the system is effected by one or several pumps connected to the distributor and controlled by the computer. The distributor is connected to the storage tanks for the reaction solutions. The valves control the supply of reaction solutions. The control of the valves can take place e.g. by motors, hydraulically or electronically and is controlled by the principal computer.

Depending on the embodiment (compare Example of Dye, Colour coding), either four NT*s or two NT*s are added simultaneously into the incorporation reaction, or only one NT*. Exemplary embodiments are indicated in FIGS. 6d and 6e.

In one embodiment (FIG. 6f), and optical detector for the control of the solution replacement is integrated. This detector is incorporated into the control circuit of the reaction platform and can control the replacement of solution e.g. by detecting the changes in the solution flowing through (e.g. optical density, light absorption or fluorescence).

If necessary, further modifications of the distributor (additional feed hoses, pumps, valves etc) can be effected to allow other accompanying steps of NAC sequencing to take place automatically.

Reaction Surface

The reaction surface is preferably situated on the underside of the cover plate, facing the lens system, of the NFC. The reaction surface is plane such that signals of many individual molecules fixed on this surface are situated within the depth of focus (focus plane) of the lens system used. The number of signals simultaneously detected by one object field are preferably numbered more than 100, more preferably more than 1000.

In a preferred embodiment, the reaction surface consists of a solid phase, e.g. glass or plastic (e.g PMMA) or silicone derivatives permeable to the excitation and fluorescent light. In another preferred embodiment, the reaction surface is the surface of a gel, e.g. a polyacrylamide gel. The gel rests on a solid substrate, e.g. glass or plastic permeable to the excitation and fluorescent light.

The NACs to be sequenced are fixed to this surface in the form of NAC primer complexes or NACF primer complexes, compare Example (Immobilisation). The immobilisation density of the NAC primer complexes or NACF primer complexes allows the identification of an individual labelled incorporated NT molecule on the surface. Preferably, NAC primer complexes or NACF primer complexes are immobilised in a density which allows the detection of at least 10 to 100 signals per 100 μm² of individually incorporated NT*s and/or at least 50%, ideally 90%, of the identified fluorescence signals originating from individual dye molecules being bound to the NT*s incorporated into NACs.

Preferably, the reaction surface carries a pattern suitable for adjusting the image. This pattern consists of microparticles, for example, with a diameter of less than 1 μm, which are fixed to the reaction surface. An example of such a pattern consists of ink particles which are fixed on the surface and have a diameter of less than 1 μm. The density of distribution of these particles is preferably less than or equal to 1 particle per 100 μm². These particles serve, firstly, for adjusting the focus plane and, secondly, to adjust images (fluorescence images) from different cycles of the sequencing reaction (compare Example of Detection).

In one embodiment, microparticles are capable of absorbing light and are made visible in the light of transmission. In another embodiment, microparticles are capable of fluorescing and are made visible e.g. in the epifluorescence mode. Irrespective of the embodiment, these particles must not interfere with the reaction and detection of the fluorescence signals of individual incorporated NT*s.

### 3b. SEQUENCE OF INDIVIDUAL STEPS IN THE AUTOMATED SEQUENCING DEVICE

The analysis of the sequences consists of the following essential steps:
a) Sample preparation
b) Immobilisation of NACs and/or NACFs
c) Cyclic steps
d) Signal analysis

The sample preparation takes place outside of the automated sequencing device and is described in the example “Sample Preparation”. The NACs or NACFs prepared for the sequencing reaction preferably have a length of between 50 and 5,000 NT and contain one PBS.

Steps b, c and d are carried out by the automated sequencing device.

Immobilisation of NACs and/or NACFs:

The aim consists of binding the NACs and/or NACFs in the form of NAC primer complexes and/or NACF primer complexes to the surface. This can take place by various processes. Some examples of fixing of complexes are indicated in the example (Immobilisation).

Cyclic Steps:

The sequence of the cyclic steps can differ, depending on the embodiment. In principle, the following steps are carried out in one cycle:
a) Addition of a reaction solution with labelled nucleotides (NT*s) and polymerase to the immobilised nucleic acid chains,
b) Incubation of immobilised nucleic acid chains with this solution under conditions suitable for extending the complementary strands by one NT,
c) Washing,
d) Detection of the signals of individual modified NT* molecules incorporated into the newly synthesised strands,
e) Removal of the label and the groups leading to termination from the incorporated nucleotides,
f) Washing.

To avoid non-specific binding of individual components of the reaction mixture, one or several blocking solutions can be applied to the surface.

Signal Analysis:

The relative position of individual NACs and/or NACFs on the reaction surface and the sequence of these NACs and/or NACFs are determined by specific allocation of the fluorescence signals detected in stage d) in successive cycles at the positions concerned. This signal analysis and sequence reconstruction can be carried out in parallel to biochemical reactions and detection or on completion of the cyclic step. An example of the operating principle of a program for signal analysis is indicated in the example “Detection”.

The execution of the cyclic step is controlled by the principal computer.

Irrespective of the sequencing process to be used, e.g. Tcherkassov et al (“Verfahren zur Bestimmung der Genexpression” (Process for the determination of gene expression) DPuMA file number 101 20 798.0-41, “Verfahren zur Analyse von Nukleinsäureketten” (Process for the analysis of nucleic acid chains) DPuMA file number 101 20 797.2-41, “Verfahren zur Analyse von Nukleinsäurekettensequenzen und der Genexpression” (process for the analysis of nucleic acid chain sequences and gene expression) DPuMA file number 101 42 256.3), the selection of the dyes depends on the filter system in the automated sequencing device. Some possible variations of colour codings are illustrated in the example (Dyes).

In one embodiment, the four NT*s can be labelled with four different but specific dyes (e.g. Cy2, Cy3, Cy5, Cy7). In this case, the reaction solution contains all four NT*s. They are incorporated in step (b) and, correspondingly, form four different signal populations on the surface. To detect the signals, the automated sequencing device is equipped, in this embodiment, with sets of filters which allow the selection of the excitation and fluorescent light of four NT*s. For example, a detection device is used in the following which is capable of differentiating only grey stage signals such that colour coding of the NT*s takes place by the defined combinations of sets of filters.

The signal detection in each cycle takes place by scanning of the surface. In this case, the reaction platform with the reaction surface is moved by the translation device (scanning table) in the X, Y, Z axis (the X, Y axis serves the purpose of changing the position, the Z axis for adjusting the focus plane, compare Example of Detection). Scanning is carried out such that several fields on the surface are examined in succession in one cycle, several signals of individual incorporated NT*s being detected per field (5000, for example). Preferably, these fields represent non-overlapping fields (FIG. 7). In all cycles, the same fields are examined. The number of fields to be examined depends on the total number of sequences which need to be analysed and differs depending on the task in hand, compare Example Sequencing, Gene expression.

In a cycle, each field is illuminated with excitation light for a specific dye selectively through the corresponding set of filters. The fluorescence signals of the incorporated NT*s are detected by means of the detection device such that one 2D image is formed per type of nucleotide and object field. Since the four NT*s carry different labels, each object field needs to be exposed to light in combination with four sets of filters in succession such that four 2D images are formed of each object field with a certain set of filters, respectively. These images carry the information on the x, y distribution of the signals of incorporated NT*s. An example of a program for the image evaluation and signal recognition is described in the example Detection.

In another embodiment, two reaction platforms are operated in parallel with one MFC, MFC1 and MFC2 each. This allows time-consuming parts of a cycle to be carried out in parallel: whereas, in MFC1, steps e-f of cycle n or steps a-c of cycle n+l are carried out, step d, scanning of the reaction surface, is carried out in MFC2. Then MFC1 and MFC2 change their positions and the reaction surface of MFC1 is scanned while the biochemical reactions are carried out in MFC2.

In another embodiment, four NT*s are labelled with only two different dyes (e.g. CY3 and CY5) compare example (Dyes). In cycle N, only two differently labelled NT*s are used simultaneously. In the next cycle N+1, the remaining two differently labelled NT*s are used correspondingly. In this embodiment, a sequencing device with only two different colour filters can be used. Other combinations of dyes, sets of filters and scanning of the surface and process control ought to appear obvious to a person skilled in the art.

In one embodiment, the reaction surface is scanned before the first cycle and each potential object field is placed into focus, the Z axis parameters for setting each object field into focus being stored by the software. In the following cycles, the stored Z axis parameters for each object field are used in each detection step.

In another embodiment, focusing of each object field takes place during the first cycle, the stored Z axis parameters for each object field being used in subsequent cycles.

In one embodiment, a control of the Z axis setting of the reaction surface is carried out in each cycle on the object field before the detection of the signals of individual molecules (compare Example of Detection). Such a control guarantees that incorporated NT*s are situated in the focus plane of the lens system and recorded clearly. This control is carried out immediately after a new field is set and, if the surface is outside of the focus plane, the autofocus function of the software is activated by the Z drive (of the scanning table built into the microscope stand or piezo drive of the lens system, for example) and the surface is brought into focus. This control is carried out on each object field once before recording the signals of individual molecules. By means of this controlled Z position, all images in this field can be taken in one cycle.

According to one embodiment, an adjustment image is taken on each field to control the X, Y axis setting of the reaction surface. An adjustment image can be taken by using a pattern described in the Example of Detection.

Principles of the X, Y, Z setting of an object field are illustrated in one embodiment shown in the example Detection.

### 4. EXAMPLES

### 4.1 Selection and Preparation of Material

### Example 4.1.1

### Selection and Preparation of Material during Sequencing of Long NACs NACs

It is possible to analyse pre-selected DNA sequences (e.g. in YAC, PAC or BAC vectors (R. Anand et al. NAR 1989 volume 17 page 3425, H. Shizuya et al. PNAS 1992 volume 89 page 8794, “Construction of bacterial artificial chromosome libraries using the modified PAC system” in “Current Protocols in Human genetics” 1996 John Wiley & Sons Inc.) cloned sections of a genome as well as non-preselected DNA (e.g. genomic DNA, cDNA, mixtures).

By way of a preliminary selection it is possible to sift out, a priori, relevant information such as sequence sections of a genome or populations of gene products, from the large quantity of genetic information and to consequently restrict the quantity of sequences to be analysed.

Preferably NACs obtained are used further without amplification (e.g. no PCR and no cloning).

The aim of the material preparation is to obtain bound single-strand NACFs with a length of preferably 50-1,000 NTs, a single primer binding site and a hybridised primer (bound NACF primer complexes). These complexes can have highly variable structures. To improve the graphicness, a few examples will now be given, the methods indicated being suitable for use individually or in combination.

Production of short nucleic acid chain fragments (50-1,000 NTs) (fragmentation step); this step is preferably carried out outside of the automatic sequencing device:

It is important for the fragmentation of the NACs to take place in such a way that fragments are obtained which represent the overlapping partial sequences of the overall sequence. This is achieved by processes in which fragments of different lengths are formed as cleavage products in random distribution.

The formation of the nucleic acid chain fragments (NACFs) can take place by several methods, e.g. by fragmenting the starting material by ultrasound or by endonucleases (“Molecular cloning” 1989 J. Sambrook et al. Cold Spring Harbor Laboratory Press), such as by non-specific endonuclease mixtures. According to the invention, ultrasound fragmentation is preferred. The conditions can be adjusted such that fragments with an average length of 100 bp to 1 kb are formed. These fragments are subsequently filled at their ends by the Klenow fragment (E. coli polymerase I) or by T4-DNA polymerase (“Molecular cloning” 1989 J. Sambrook et al. Cold Spring Harbor Laboratory Press).

Also, complementary short NACFs can be synthesised from long NACs using randomised primers. This method is particularly preferred for the analysis of the gene sequences. In this process, single-strand DNA fragments are formed with randomised primers and a reverse transcriptase on the mRNA (Zhang-J et al. Biochem. J. 1999 volume 337 page 231, Ledbetter et al. J. Biol. Chem. 1994 volume 269 page 31544, Kolls et al. Anal. Biochem. 1993 volume 208 page 264, Decraene et al. Biotechniques 1999 volume 27 page 962).

Introduction of a Primer Binding Site into the NACFs:

The primer binding site (PBS) is a sequence section which is to allow selective binding of the primer to the NACF.

According to one embodiment, the primer binding sites can be different such that several different primers need to be used. In this case, certain sequence sections of the overall sequence can serve as natural PBSs for specific primers. This embodiment is particularly suitable for investigating SNP sites.

According to another embodiment, it is advantageous for reasons of simplification of the analysis, for a uniform primer binding site to be present in all NACFs. According to a preferred embodiment of the invention, the primer binding sites are therefore introduced additionally into the NACFs. In this way, primers with a uniform structure can be used for the reaction.

In the following, this embodiment will be described in detail.

The composition of the primer binding site is not restricted. Preferably, its length is between 20 and 50 NTs. The primer binding site may carry a functional group for the immobilisation of the NACF. This functional group may consist of a biotin group, for example.

The ligation and the nucleotide tailing to DNA fragments will be described in the following as an example of the introduction of a uniform primer binding site.

A) Ligation

In this case, a double-stranded oligonucleotide complex with one primer binding site is used. This is ligated to the DNA fragments by means of commercially available ligases (“Molecular cloning” 1989 J. Sambrook et al. Cold Spring Harbor Laboratory Press). It is important for only a single primer binding site to be ligated to the DNA fragment. This is achieved by modifying one side of the oligonucleotide complex on both strands, for example. The modifying groups on the oligonucleotide complex can be used for immobilisation. The synthesis and modification of such an oligonucleotide complex can be carried out according to standardised specifications. The DNA synthesiser 380 A Applied Biosystems, for example, can be used for the synthesis. However, oligonucleotides with a certain composition with or without modification are also commercially available as toll synthesis systems, e.g. from MWG-Biotech GmbH, Germany.

b) Nucleotide Tailing

Instead of ligation with an oligonucleotide, it is possible to attach several (e.g. between 10 and 20) nucleoside monophosphates to the 3′ end of an ss-DNA fragment by means of a terminal deoxynucleotidyl transferase (“Molecular cloning” 1989 J. Sambrook et al. Cold Spring Harbor Laboratory Press, “Method in Enzymology” 1999 volume 303 page 37-38) (FIG. 4) e.g. several guanosin monophosphates (called (G)n tailing). The fragment formed is used to bind the primer, in this example a (C) n primer.

Preparation of the Single Strand

Single-strand NACFs are required for the sequencing reaction. If the starting material is present in the double-stranded form, there are several possibilities for producing a single-strand form from double-stranded DNA (e.g. heat denaturing or alkali denaturing) (“Molecular cloning” 1989 J. Sambrook et al. Cold Spring Harbor Laboratory Press).

### Example 4.1.2

### Material Selection and Preparation for Gene Expression Analysis

Gene products may originate from different biological objects such as individual cells, cell populations, a tissue or complete organisms. Biological fluids such as blood, sputum or liquor can also be used as a source of gene products. The method of obtaining gene products from the different biological objects can be found in the following literature sources, for example: “Molecular cloning” 1989, Ed. Maniatis, Cold Spring Harbor Laboratory, “Method in Enzymology” 1999, volume 303, “cDNA library protocols” 1997, Ed. I. G. Cowell, Humana Press Inc.

Both the entirety of the isolated gene products and parts thereof selected by preliminary selection can be used in the sequencing reaction. By way of the preliminary selection, the quantity of the gene products to be analysed can be reduced. The preliminary selection can take place by molecular biological processes, for example, such as e.g. PCR amplification, gel separation or hybridisation with other nucleic acid chains (“Molecular cloning” 1989, Ed. Maniatis, Cold Spring Harbor Laboratory, “Method in Enzymology” 1999, volume 303, “cDNA library protocols” 1997, Ed. I. G. Cowell, Humana Press Inc.)

The gene products in their entirety are preferably used as starting material.

Preferably, gene products are continued to be used further without amplification steps (e.g. no PCR and no cloning).

The aim of the preparation of the material is to form extensible gene product primer complexes bound to the surface, from the starting material. Only one primer at most ought to bind per gene product in this respect.

Primer Binding Site (PBS):

Each gene product preferably has only one primer binding site.

A primer binding site is a section of a sequence which is to permit a selective binding of the primer to the gene product.

Sections in the nucleic acid sequence which naturally occur in the sequences to be analysed can serve as primer binding sites (e.g. poly-A stretches in the mRNA). A primer binding site can also be introduced additionally into the gene product (“Molecular cloning” 1989, Ed. Maniatis, Cold Spring Harbor Laboratory, “Method in Enzymology” 1999, volume 303, “cDNA library protocols” 1997, Ed. I. G. Cowell, Humana Press Inc.)

For reasons of simplification of the analysis, it may be important that a primer binding site is present in all gene products which is as uniform as possible. In this case, primers with a uniform structure can be used in the reaction. The composition of the primer binding site is not restricted. Preferably, its length is between 10 and 100 NTs. The primer binding site may carry a functional group, e.g. for binding the gene product to the surface. This functional group may consist e.g. of a biotin or a digoxigenin group.

As an example of the insertion of a primer binding site into the gene product, nucleotide tailing of antisense cDNA fragments will now be described.

Firstly, single-strand cDNAs are synthesised from mRNAs. This results in a population of cDNA molecules which represent a copy of the mRNA population, the so-called antisense cDNA. “Molecular cloning” 1989, Ed. Maniatis, Cold Spring Harbor Laboratory, “Method in Enzymology” 1999, volume 303, “cDNA library protocols” 1997, Ed. I. G. Cowell, Humana Press Inc.) By means of a terminal deoxynucleotidyl transferase, it is possible to attach several (e.g. between 10 and 20) nucleoside monophosphates to the 3′ end of this antisense cDNA, e.g. several adenosin monophosphates (referred to as (dA)n tail). The fragment formed is used to bind the primer, in this example a (dt)n primer (“Molecular cloning” 1989, J. Sambrook et al. Cold Spring Harbor Laboratory Press, “Method in Enzymology” 1999, volume 303, page 37-38).

### Example 4.1.3

### Primer for the Sequencing Reaction

This has the task of allowing the start to take place at a single site of the NAC or NACF. Preferably, it binds to the primer binding site in the NAC (e.g. in the oligonucleotide or in the gene product) or in the NACF. The composition and length of the primer are not restricted. Apart from the starting function, the primer can also take on other functions such as creating a link to the reaction surface. Primers should be adjusted to the length and composition of the primer binding site such that the primer allows the start of the sequencing reaction with the polymerase concerned.

When using different, e.g. primer binding sites naturally occurring in the original overall sequence, primers are used which are sequence-specific for the primer binding site concerned. In this case, a primer mixture is used for sequencing.

In the case of a uniform primer binding site, e.g. one coupled to the NACFs by ligation, a uniform primer is used.

Preferably, the length of the primer is between 6 and 100 NTs, optimally between 15 and 30 NTs. The primer can carry a functional group which is used to immobilise the NACF; such a functional group consists of a biotin group, for example (compare chapter on Immobilisation). It should not interfere with sequencing. The synthesis of such a primer can be carried out e.g. with the DNA synthesiser 380 A Applied Biosystems or it can be carried out as a toll synthesis by a commercial provider, e.g. MWG-Biotech GmbH, Germany.

Prior to hybridisation, the primer can be fixed to the surface of the NACs or NACFs to be analysed by using different techniques or synthesised directly on the surface, e.g. according to (McGall et al. U.S. Pat. No. 5,412,087, Barrett et al. U.S. Pat. No. 5,482,867, Mirzabkov et al. U.S. Pat. No. 5,981,734, “Microarray biochip technology” 2000 M. Schena Eaton Publishing, “DNA Microarrays” 1999 M. Schena Oxford University Press, Fodor et al. Science 1991 volume 285 page 767, Timofeev et al. Nucleic Acid Research (NAR) 1996, volume 24 page 3142, Ghosh et al. NAR 1987 volume 15 page 5353, Gingeras et al. NAR 1987 volume 15 page 5373, Maskos et al. NAR 1992 volume 20 page 1679).

The primers are bound to the surface in a density of between 10 to 100 per 100 μm², 100 to 10,000 per 100 μm² or 10,000 to 1,000,000 per 100 μm². A greater fixing density is preferred, no need for optical identification of each primer arising: greater primer densities accelerate hybridisation of the NACs or NACFs to be analysed.

The primer or the primer mixture is incubated with NACFs under hybridisation conditions which allow it to bind selectively to the primer binding site of the NACs or the NACFs. This primer hybridisation (annealing) can be carried out before (1), during (2) or after (3) the binding of the NACs or NACFs to the surface. The optimisation of the hybridisation conditions depends on the precise structure of the primer binding site and the primer itself and can be calculated according to Rychlik et al. (NAR 1990 volume 18 page 6409). In the following, these hybridisation conditions will be referred to as standardised hybridisation conditions.

If a primer binding site of known structure common to all NACs and/or NACFs is introduced e.g. by ligation, primers with a uniform structure can be used. The primer binding site can carry a functional group at its 3′-end, which functional group serves the purpose of immobilisation, for example. This group may be a biotin group, for example. The primer has a structure complementary to the primer binding site.

Binding of primers to the surface of the MFC takes place prior to experiments and preferably does not form part of the process. Chips with primers bound to the surface of the MFC can be stored for prolonged periods.

### Example 4.1.4

### Immobilisation

Fixing of NAC primer complexes or NACF primer complexes to the surface (binding and/or immobilisation of gene products):

It is the aim of the fixing operation (immobilisation) to fix NAC primer complexes or NACF primer complexes on a suitable plane surface in such a way that a cyclic enzymatic sequencing reaction can take place. This may, for example, occur by binding the primer (compare above) or the NACs or NACFs to the surface.

The sequence of the steps for fixing NAC primer complexes or NACFs primer complexes can vary:

1) The complexes may first be formed by hybridisation (annealing) in a solution and subsequently be bound to the surface.

2) Primers can first be bound to a surface and subsequently NACs or NACFs can be hybridised to the bound primers, NACFs primer complexes, for example, being formed (NACFs bound indirectly to the surface).

3) The NACs or NACFs can be bound first to the surface (NACFs bound directly to the surface) and the primers hybridised in the subsequent step to the bound NACs or NACFs, NAC primer complexes or NACFs primer complexes being formed.

The immobilisation of the NACs or NACFss to the surface can consequently take place by direct or indirect binding.

In a preferred embodiment, the reaction surface forms part of the MFC, the material of the surface being permeable to electromagnetic radiation (excitation and fluorescent light). Moreover, this material is inert vis-à-vis enzymatic reactions and causes no interference with detection. Glass or plastics (e.g. PMMA) or any other material satisfying these functional requirements can be used. Preferably, the reaction surface is not deformable; otherwise, a distortion of the signals can be expected during repeated detection.

If a gel type solid phase (surface of a gel) is used, this gel can be e.g. an agarose or polyacrylamide gel. Preferably, the gel is freely penetrable by molecules with a molecular weight of less than 5,000 Da (for example, a 1 to 2% agarose gel or 5 to 15% polyacrylamide gel can be used). Compared with other solid reaction surfaces, such a gel surface has the advantage that much less non-specific binding of NT*s to the surface occurs. By binding the NACFs primer complexes to the surface, the detection of the fluorescence signals of incorporated NTs* is possible. The signals of free NTs* are not detected because they do not bind to the material of the gel and are thus not immobilised. Preferably, the gel is fixed to a solid substrate. This solid substrate can consist of glass or plastics (e.g. PMMA).

Preferably, the thickness of the gel is not more than 0.1 mm. Preferably, however, the thickness of the gel is greater than the simple depth of focus of the lens system so that NTs* non-specifically bound to the solid substrate do not reach the focus plane and are thus detected. If the depth of focus is e.g. 0.3 μm, the gel thickness is preferably between 1 μm and 100 μm. The surface can be produced as a continuous surface or as a discontinuous surface composed of individual small components (e.g. agarose beads). The reaction surface must be large enough to be able to immobilise the necessary number of complexes with a corresponding density. Preferably, the reaction surface should not be greater than 20 cm².

If the NACF primer complexes are fixed on the surface via the NACFs, this can take place by binding the NACFs to one of the two chain ends, for example. This can be achieved by corresponding covalent, affine or other bonds. Numerous examples of the immobilisation of nucleic acids are known (McGall et al. U.S. Pat. No. 5,412,087, Nikiforov et al. U.S. Pat. No. 5,610,287, Barrett et al. U.S. Pat. No. 5,482,867, Mirzabkov et al. U.S. Pat. No. 5,981,734, “Microarray biochip technology” 2000 M. Schena Eaton Publishing, “DNA Microarrays” 1999 M. Schena Oxford University Press, Rasmussen et al. Analytical Biochemistry volume 198, page 138, Allemand et al. Biophysical Journal 1997, volume 73, page 2064, Trabesinger et al. Analytical Chemistry 1999, volume 71, page 279, Osborne et al. Analytical Chemistry 2000, volume 72, page 3678, Timofeev et al. Nucleic Acid Research (NAR) 1996, volume 24 page 3142, Ghosh et al. NAR 1987 volume 15 page 5353, Gingeras et al. NAR 1987 volume 15 page 5373, Maskos et al. NAR 1992 volume 20 page 1679). Fixing can also be achieved by a non-specific binding such as e.g. by drying out of the sample containing NACFs on the plane surface. The same applies also to NACs.

The NACs and/or NACFs are bound on the surface e.g. in a density of between 10 and 100 NACs and/or NACFs per 100 μm², 100 to 10,000 per 100 μm², 10,000 to 1,000,000 per 100 μm².

The density of extensible NAC primer complexes and/or NACF primer complexes, which is necessary for detection, is approximately 10 to 100 per 100 μm². It can be achieved before, during or after the hybridisation of the primers to the gene products.

As an example, some methods for binding NACF primer complexes are illustrated in further detail in the following: According to one embodiment, immobilisation of the NACFs is effected via biotin-avidin or biotin-streptavidin binding. In this case, avidin or streptavidin is covalently bound on the surface, the 5′ end of the primer contains biotin. Following hybridisation of the labelled primers with NACFs (in solution), these are fixed on the surface coated with avidin/streptavidin. The concentration of the hybridisation products labelled with biotin and the time of incubation of this solution with the surface is selected in such a way that a density suitable for sequencing is achieved already in this step.

In another preferred embodiment, the primers suitable for the sequencing reaction are fixed on the surface by suitable methods (compare above) before the sequencing reaction. The single-strand NACs or NACFs with one primer binding site per NAC or NACF are incubated (annealed) with these primers under hybridisation conditions. As a result, they bind to the fixed primers and are thus bound (indirect binding), primer NAC complexes or primer NACF complexes being formed. The concentration of the single-strand NACs or NACFs and the hybridisation conditions are chosen such that an immobilisation density suitable for sequencing of approx. 10 to 100 extensible complexes per 100 μm² is obtained. After hybridisation, the non-bound NACFs are removed by a wash step. In the case of this embodiment, a surface with a high primer density is preferred, e.g. approx. 1,000,000 primers per 100 μm² or higher since the desired density of NAC primer complexes or NACF primer complexes is achieved more rapidly, the NACs or NACFs binding only to part of the primer.

In another embodiment, the NACs or NACFs are directly bound to the surface (see above) and subsequently incubated with primers under hybridisation conditions. At a density of approximately 10 to 100 NACs or NACFs per 100 μm², an attempt will be made to provide all available NACs or NACFs with a primer and to make them available for the sequencing reaction. This can be achieved e.g. by a high primer concentration (concentration of the primers as a whole), for example 1 to 10 mmole/l. At a higher density of the fixed NACs or NACFs on the surface, for example 10.000 to 1,000,000 per 100 μm², the density of the complexes necessary for optical detection can be achieved during primer hybridisation. In this case, the hybridisation conditions (e.g. temperature, time, buffer, primer concentration) must be selected such that the primers bind only to a part of the immobilised NACs or NACFs.

If the surface of a solid phase (e.g. silicone or glass) is to be used for immobilisation, a blocking solution is preferably applied to the surface before step (a) in each cycle which solution serves the purpose of avoiding a non-specific adsorption of NTs* to the surface.

### 4.2 The Example of Reaction Solutions

Solution A: 50 mM phosphate buffer, pH 8.5, 10% glycerine, 5 mM MG²⁺, 1 mM Mn²⁺.

Solution B (reaction solution NT*(n)): solution A, polymerase, labelled NT*(n)

Solution C (cleavage solution): solution A, cleavage reagents

Solution D, the sample to be analysed in solution A

Solution E (wash solution) is the same as solution A

Solution F, 1 mg/ml acetylated BSA in solution A (a blocking solution for the reduction of the non-specific binding of the NT*s to the solid surface such as glass, silicon etc).

### 4.3 Example of Dyes

Label, Fluorescent Dye

Each base is labelled with a characteristic label (F). The label is a fluorescent dye. Several factors influence the selection of the fluorescent dye. The selection is not limited, provided the dye satisfies the following requirements:

a) The detection device used must be able to identify the label as a single molecule bound to a DNA under mild conditions (preferably reaction conditions). Preferably, the dyes have a high photostability. Preferably, their fluorescence is quenched by DNA either not at all or only insignificantly.

b) The dye bound to the NT must not cause any irreversible interference with the enzymatic reaction.

c) NTs* labelled with the dye must be incorporated by the polymerase into the nucleic acid chain.

d) During labelling with different dyes, these dyes should not exhibit any major overlap regarding their emission spectra.

Fluorescent dyes suitable for use in connection with the present invention are compiled in “Handbook of Fluorescent Probes and Research Chemicals” 6^{th} ed. 1996, R. Haugland, Molecular Probes. According to the invention, the following dye classes are preferably used as labels: cyanine dyes and their derivatives (e.g. Cy2, Cy3, Cy5, Cy7 Amersham Pharmacia Biotech, Waggoner U.S. Pat. No. 5,268,486), rhodamines and their derivatives (e.g. TAMRA, TRITC, RG6, R110, ROX, Molecular Probes, compare handbook), xanthene derivatives (e.g. Alexa 568, Alexa 594, Molecular Probes, Mao et al. U.S. Pat. No. 6,130,101). These dyes are commercially available.

In this respect, corresponding dyes can be selected according to the spectral properties and the equipment available. The dyes are coupled to the linker e.g. via thiocyanate or ester bonds (“Handbook of Fluorescent Probes and Research Chemicals” 6^{th} ed. 1996, R. Haugland, Molecular Probes, Jameson et al. Methods in Enzymology 1997 volume 278 page 363, Waggoner Methods in Enzymology 1995 volume 246 page 362), compare also the patent applications of Tcherkassov et al (“Verfahren zur Bestimmung der Genexpression” (Process for the determination of gene expression) DPuMA file number 101 20 798.0-41, “Verfahren zur Analyse von Nukleinsäureketten” (Process for the analysis of nucleic acid chains) DPuMA file number 101 20 797.2-41, “Verfahren zur Analyse von Nukleinsäurekettensequenzen und der Genexpression” (Process for the analysis of nucleic acid chain sequences and gene expression) DPuMA file number 101 42 256.3).

Coloured Coding Scheme, Number of Dyes (Colour Coding)

A cycle can be carried out with:
a) four differently labelled NT*s
b) two differently labelled NT*s
c) one labelled NT*
d) two differently labelled NT*s and two non-labelled NTs,
i.e.

a) All four NTs can be labelled with different dyes and all 4 NT*s used simultaneously in the reaction. In this way, the sequencing of a nucleic acid chain is achieved with a minimal number of cycles. However, this variant of the invention makes very high demands on the detection system: 4 different dyes need to be identified in each cycle.

b) To simplify the detection, a labelling with two dyes can be chosen. In this case, 2 pairs of NTs* are formed which are differently labelled in each case, e.g. A and G carry the label “X”, C and U carry the label “Y”. Two differently labelled NTs* are used simultaneously in the reaction in one cycle (n), e.g. C* in combination with A* and U* and G* are then added in the subsequent cycle (n+1).

c) It is also possible to use only a single dye to label all 4 NTs* and to employ only one NT* per cycle.

d) In a technically simplified embodiment, two differently labelled NT*s and two non-labelled NTs (so-called 2NT*s/2NTs method) are used per cycle. This embodiment can be used in order to determine variants (e.g. mutations or alternatively spliced genes) of a sequence which is already known.

Other combinations are obvious.

### 4.4 Example of Detection

1) Preparation for detection

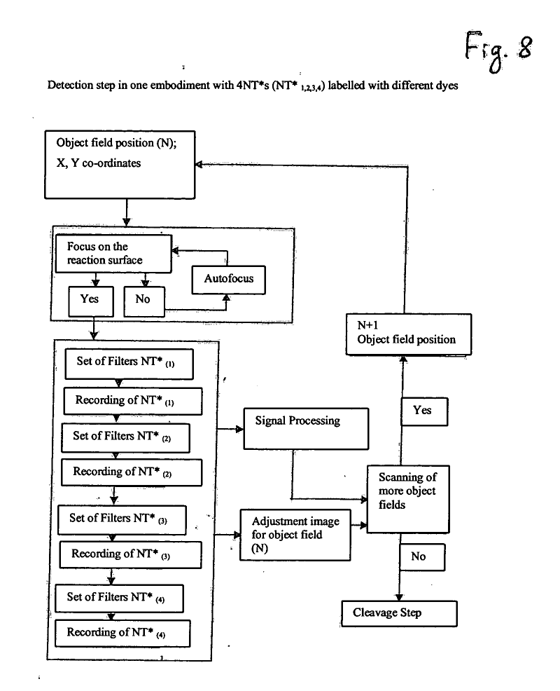

2) Execution of a detection step in each cycle. The diagram in FIG. 8 represents, in the form of an example, the course of detection on an object field, 4 NT*s (NT*_{1,2,3,4}) being labelled with different dyes and incorporated into the immobilised NACs in one reaction. Each detection step is carried out as a scanning process comprising the following operations:
a) Setting of the position of the lens system (X, Y axis)
b) Setting of the focus plane (Z axis)
c) Detection of the signals of individual molecules, allocation of the signal to NT* and allocation of the signal to the NAC or NACF concerned
d) Displacement to the next position on the surface

The signals of NTs* incorporated into the NACs or NACFs are recorded by scanning the surface. In this case, the lens system is moved over the surface in a stepwise movement (FIG. 7) such that a two dimensional image is formed of every surface position (2D image).

1) Preparation for detection

To begin with, it is determined how many NACFs need to be analysed to reconstruct the original sequence during sequencing of long nucleic acid chains (e.g. DNA segments 1 Mb in length). In the case of a reconstruction according to the shotgun process (“Automated DNA sequencing and analysis” page 231 ff. 1994 M Adams et al. Academic Press, Huang et at. Genom Res. 1999 volume 9, page 868, Huang Genomics 1996 volume 33, page 21, Bonfield et al. NAR 1995 volume 23, page 4992, Miller et al. J. Comput. Biol 1994 volume 1, page 257) the following factors play a part:

1) A sequence of approximately 300-500 NTs is determined for each NACF during sequencing.

2) The overall length of the sequence to be analysed is important.

3) A certain level of redundancy needs to be achieved during sequencing in order to increase the accuracy and to correct possible errors.

Overall, an approximately 10-100 fold quantity of raw sequences is required for the reconstruction of the major part of the original sequence, i.e. in this example with one Mb, 10 to 100 Mb of raw sequence data are required. With an average sequence length of 400 bp per NACF, 25,000 to 250,000 DNA fragments are consequently required.

During the analysis of gene expression, it is determined how many copies of the gene products are required for expression analysis. Several factors play a part in this. The exact number depends e.g. on the relative presence of the gene products in the batch and on the desired accuracy of the analysis. The number of analysed gene products is preferably between 1,000 and 10,000,000. For strongly expressed genes, the number of analysed gene products can be low, e.g. 1,000 to 10,000. During the analysis of weakly expressed genes, it must be increased, e.g. to 100,000 or more.

For example, 100,000 individual gene products are analysed simultaneously. In this case, weakly expressed genes (e.g. with approximately 100 mRNA molecules/cell, corresponding to approximately 0.02% of total mRNA) are represented in the reaction by an average of 20 identified gene products.

Determination of the total number (N_{OF}) of the object fields which need to be scanned:

The number (N_{NAC}) of the NACs/NACFs to be analysed in conjunction with the average density of the NACs/NACFs participating in the sequencing reaction per object field (D) determines the number (N_{OF}) of object fields which need to be scanned. The principal computer calculates this N_{OF} during the first cycle.

2) The execution of a detection step in each cycle is explained by way of the example of sequencing of a long nucleic acid chain.

For sequencing, the X, Y positions of the NACFs on the surface need to be determined in order to obtain a basis for the allocation of the signals. Knowing these positions makes it possible to provide an indication as to whether the signals of individual molecules originate from incorporated NTs* or from NTs* randomly bound to the surface. These X, Y positions can be identified by different methods.

In a preferred embodiment, the X, Y positions of immobilised NACFs are identified during sequencing. In this case, use is made of the fact that the signals from the NTs* incorporated into the nucleic acid chain always have the same co-ordinates. This is guaranteed by fixing the nucleic acid chains. The non-specifically bound NTs* bind randomly to different sites of the surface.

To identify the X, Y positions of fixed NACFs, the signals are examined for agreement between their co-ordinates from different cycles occurring in succession. This can be done e.g. at the beginning of sequencing. The agreeing co-ordinates are evaluated as co-ordinates of DNA fragments and stored.

The scanning system must be capable of scanning the surface reproducibly over several cycles. X, Y and Z axis settings at each surface position can be verified by a computer. The stability and reproducibility of the setting of lens system positions in each scanning process are decisive for the quality of detection and consequently the identification of the signals of individual molecules.

a) Setting of the position of the lens system (X, Y axis). The mechanical instability of the commercially available scanning tables and the poor reproducibility of the repeated settings of the same X, Y positions make it difficult to carry out accurate analyses of the signals of individual molecules over several cycles. There are many possibilities for improving the agreement between co-ordinates during repeated setting operations and/or verifying possible deviations. One verification method is provided here as an example. Following rough mechanical setting of the position of the lens system, a control image is taken of a pattern firmly connected to the surface. Even if the mechanical setting does not have precisely the same co-ordinates (deviations of up to several μm are possible over several cycles), it is possible to effect a correction by means of an optical control. The control image of the pattern is used as a system of co-ordinates for the image with signals of incorporated NTs*. A precondition for such a correction is that no further movements of the surfaces take place in the interval between two images being taken. A relationship is established between signals of individual molecules and the pattern such that an X, Y deviation in the pattern position means an identical X, Y deviation in the position of the signals of individual molecules. The control image of the pattern can be taken before, during or after the detection of individual molecules. Such a control image must correspondingly be taken for each setting on a new surface position.

b) Setting of the focus plane (Z axis)

The surface is not absolutely plane and exhibits different unevennesses. This changes the distance between the surface and the lens system during scanning of adjacent sites. These differences in the distance can lead to individual molecules leaving the focus plane thus escaping detection.

For this reason it is important that the focus plane is correctly adjusted during scanning of the surface before the signals of individual molecules on each object field are recorded. This is preferably done by setting the focus plane to a certain pattern which is firmly fixed to the reaction surface. This pattern can be formed by particles with a diameter of approximately 1 μm, for example. These particles can be visualised e.g. by the backlighting mode, for example. Subsequently, a changeover to the fluorescence mode is effected and signals of individual molecules are detected.

According to one embodiment, the visualisation of the setting pattern is effected by illumination from underneath. For this purpose, the reaction platform is provided with an aperture in its lower part such that the reaction surface can be illuminated from below e.g. by backlighting or phase contrast lighting (FIG. 4a).

In another embodiment, the setting pattern itself is able to fluorescence such that the setting pattern can be visualised in the fluorescence mode with appropriate illumination (FIG. 4b). Preferably, light of a different wavelength is used for visualising the setting pattern which light does not interfere with the detection of the signals of individual molecules.

c) Detection of the signals of individual molecules, allocation of the signal to NT* and allocation of the signal to the NAC concerned.

The two-dimensional image of the reaction surface produced by means of the detection system contains the signal information of many NT*s incorporated into the NACFs. Before further processing is carried out, they must be extracted from the total quantity of data of the image information by means of suitable methods. The algorithms for scaling, transformation and filtering of the image information, which are necessary for this purpose, belong to the standard repertoire of digital image processing and pattern recognition (“Haberäcker P. “Praxis der Digitalen Bildverarbeitung und Mustererkennung” (practice of digital image processing and pattern recognition). Hanser-Verlag, Munich, Vienna, 1995; Galbiati L. J. “Machine vision and digital image processing fundamentals”. Prentice Hall, Englewood Cliffs, N.J., 1990). The signal extraction preferably takes place via a grey-scale picture which depicts the brightness distribution of the reaction surface for the fluorescence channel concerned. If several nucleotides are used with different fluorescent dyes for the sequencing reaction, a separate grey scale picture can be produced for each fluorescence-labelled nucleotide (A, T, C, G or U). For this purpose, 2 processes can basically be used:

1. By using suitable filters (sets of Zeiss filters), a grey scale picture is produced for each fluorescence channel.

2. From a multiple channel colour image that has been taken, the relevant colour channels are extracted by means of a suitable algorithm using an image processing program and processed further individually as a grey scale picture. For the channel extraction, a colour threshold value algorithm specific for the channel concerned is used. In this way, individual grey scale pictures 1 to N are initially formed from a multi channel colour image. These pictures can be defined as follows:

| | |
|---|---|
| GB_{N} = (s (x, y)) | single channel grey scale picture |
| N = (1, . . . , number of | |
| fluorescence channels) | |
| M = (0, 1, . . . , 255) | amount of grey scale |
| S + (s (x, y)) | image matrix of the grey scale picture |
| x = 0, 1 . . . , L-1 | image lines |
| y = 0, 1, . . . , R-1 | image columns |
| (x, y) | site co-ordinates of an image point |
| s (x, y) ε M | grey scale of the image point |

By means of a suitable program, the relevant image information is extracted from this amount of data. Such a program ought to carry out the following operating steps:

Carry out for GB₁ to GB_{N}:

I. Preprocessing of the image e.g., if necessary, reduction of the image noise formed by the digitalisation of the image information, e.g. by grey scale smoothing.

II. Examination of each image point (x, y) of the grey scale picture as to whether this point exhibits the properties of a fluorescence point in connection with the adjacent image points surrounding it directly and those further removed. These properties depend, among other things, on the detection equipment used and the resolution of the grey scale picture. They can, for example, represent a typical distribution pattern of brightness intensity values over a matrix surrounding the image point. The methods of image segmentation used for this purpose extend from simple threshold value methods to the use of neuronal networks.

If an image point (x, y) satisfies these requirements, a comparison is then carried out with the co-ordinates of NACFs identified in sequencing cycles carried out so far. In the case of agreement, the allocation of the signal with the nucleotide emerging from the fluorescence channel concerned to this NACF takes place. Signals with non-agreeing co-ordinates are assessed as background signals and discarded. The analysis of the signals can take place in parallel to the scanning process.

According to an exemplary embodiment, an 8 bit grey scale picture of with a resolution of 1317×1035 pixel was used. In order to reduce the changes to the picture resulting from digitalisation, preliminary processing of the overall picture was first effected: the average value of the brightnesses of its eight neighbours was allocated to each image point. As a result, a pattern, typical of a fluorescence point, of a central image point with the highest brightness value and neighbouring image points with brightnesses decreasing towards all side is thus formed with the resolution chosen. If an image point satisfies these criteria and if the centrifugal brightness decrease exceeds a certain threshold value (to the exclusion of weak fluorescence points), this central image point is used as co-ordinate for a fluorescence point.

d) Displacement of the lens system towards the next position on the surface. After detecting the signals of individual molecules, the lens system is positioned at a different position of the surface.

Overall, a sequence of images, for example, can be taken while controlling the X, Y position, the setting of the focus plane and with the detection of individual molecules for every new position of the lens system. These steps can be controlled by means of a computer.

### 4.5 Example of Sequence Analysis

Sequence analysis with 4 labelled NT*s with, in a preferred embodiment of the invention, all four NT*s used in the reaction being labelled with four different fluorescent dyes and used simultaneously in the reaction. This embodiment can be used for the analyses detailed below, for example.

### 4.5. A Sequencing of Long Nucleic Acids

This process is based on the reconstruction of the original sequences according to the shotgun principle (“Automated DNA sequencing and analysis” page 231 ff. 1994 M Adams et al. Academic Press, Huang et at. Genom Res. 1999 volume 9, page 868, Huang Genomics 1996 volume 33, page 21, Bonfield et al. NAR 1995 volume 23, page 4992, Miller et al. J. Comput. Biol 1994 volume 1, page 257). (This principle is suitable in particular for the analysis of new unknown sequences).

Sequencing of a Long DNA Section

In the following, the sequencing of long nucleic acid chains is to be illustrated schematically by way of the sequencing of a DNA section 1 Mb in length. The sequencing is based on the shotgun principle (“Automated DNA sequencing and analysis” page 231 ff. 1994 M. Adams et al. Academic Press, Huang et at. Genom Res. 1999 volume 9, page 868, Huang Genomics 1996 volume 33, page 21, Bonfield et al. NAR 1995 volume 23, page 4992, Miller et al. J. Comput. Biol 1994 volume 1, page 257). The material to be analysed is prepared for the sequencing reaction by splitting it into fragments preferably 50 to 1,000 bp in length. Each fragment is subsequently provided with a primer binding site and a primer. This mixture of different DNA fragments is then fixed on a plane surface. The non-bound DNA fragments are removed by a wash step. Subsequently, the sequencing reaction is carried out on the entire reaction surface. To reconstruct a DNA sequence 1 Mb in length, the sequences of NACFs should preferably be longer than 300 NTs, on average approximately 400 bp. Since only one labelled NT* is incorporated per cycle, at least 400 cycles are necessary for sequencing.

In total, an approximately 10 to 100 fold quantity of raw sequences, i.e. 10 to 100 Mb, is necessary to reconstruct the original sequence. With an average sequence length of approximately 400 bp per NACF, 25,000 to 250,000 DNA fragments are consequently required in order to cover more than 99.995% of the overall sequence.

The NACF sequences determined represent a population of overlapping partial sequences which can be joined together by commercially available programs to form the overall sequence of the NAC (“Automated DNA sequencing and analysis” page 231 ff. 1994 M Adams et al. Academic Press, Huang et at. Genom Res. 1999 volume 9, page 868, Huang Genomics 1996 volume 33, page 21, Bonfield et al. NAR 1995 volume 23, page 4992, Miller et al. J. Comput. Biol 1994 volume 1, page 257).

Sequencing of the Gene Products using the Example of cDNA Sequencing

In a preferred embodiment, several sequences can be analysed in one batch instead of just one sequence. The original sequences can be reconstructed from the raw data obtained, e.g. using the shotgun principle.

First of all, NACFs are produced. It is, for example, possible to convert mRNA into a double-strand cDNA and to fragment this cDNA with ultrasound. Subsequently, these NACFs are provided with a primer binding site, denatured, immobilised and hybridised with a primer. It should be noted in the case of this variation of sample preparation that the cDNA molecules may represent incomplete mRNA sequences (Method in Enzymology 1999, volume 303, page 19 and other articles in this volume, “cDNA library protocols” 1997 Humana Press). Another possibility during the generation of single-strand NACFs of mRNA consists of the reverse transcription of the mRNA with randomised primers. During this process, many relatively short antisense DNA fragments are formed (Zhang-J et al. Biochem. J. 1999 volume 337 page 231, Ledbetter et al. J. Biol. Chem. 1994 volume 269 page 31544, Kolls et al. Anal/Biochem. 1993 volume 208 page 264, Decraene et al. Biotechniques 1999 volume 27 page 962). These fragments can subsequently be provided with a primer binding site (see above). Further steps correspond to the processes described above. By means of this method, complete mRNA sequences (from the 5′ to the 3′ end) can be analysed, since the randomised primers can bind over the entire length of the mRNA.

Immobilised NACFs are analysed by means of one of the embodiments of sequencing indicated above. Since mRNA sequences exhibit essentially fewer repetitive sequences that e.g. genomic DNA, the number of detected signals from the incorporated NTs* of one NACF can be less than 300 and is preferably between 20 and 1000. The number of NACFs which need to be analysed is calculated according to the same principles as for a shotgun reconstruction of a long sequence.

From NACF sequences, the original gene sequences are reconstructed according to the principles of the shotgun process.

This method allows the simultaneous sequencing of many mRNAs without prior cloning.

### 4.5 B Gene Expression Analysis

Sequence Analysis with 4 Labelled NT*s

In a preferred embodiment of the invention, all four NT* used in the reaction are labelled with fluorescent dyes.

For this purpose, one of the above-mentioned coloured coding schemes are used. The number of NTs determined for each sequence from a gene product is between 5 and 100, ideally between 20 and 50.

Analysis

The data obtained (short sequences) are compared with known gene sequences using a program. Such a program can be based e.g. on a BLAST or FASTA algorithm (“Introduction to computational Biology” 1995 M. A. Waterman Chapman & Hall).

By selecting the method for the preparation of the material, it is determined, among other things, in which sections of the gene products the sequences are to be determined and to which strand (sense or antisense) they belong. For example, sequences of NTRs (non-translating regions) are determined when the polyA stretches are used as primer binding site in mRNA. When using the method with antisense cDNA as matrix, the sequences determined originate, among other things, from the protein-encoding region of the gene products.

In the case of a preferred simple variant of the invention, the gene expression is determined only qualitatively. In this case, only the fact of the expression of certain genes is of importance.

In the case of another preferred embodiment, a quantitative determination of the relationships between individual gene products in the batch is of interest. It is known that the activity of a gene in a cell is represented by a population of identical mRNA molecules. In a cell, many genes are active simultaneously and are expressed with different intensities leading to the presence of many different differently strongly represented mRNA populations.

In the following, the quantitative analysis of gene expression is discussed in further detail:

For a quantitative analysis of gene expression, the abundances of individual gene products in the sequencing reaction are determined. In this respect, the products of strongly expressed genes are more frequently represented in the sequencing reaction than the weakly expressed genes.

After allocating the sequences to certain genes, the portion of sequences determined for each individual gene is determined. Genes with a strong expression have a higher portion of the overall population of the gene products than genes with a weak expression.

The number of gene products analysed is preferably between 1,000 and 10,000,000. The exact number of the gene products to be analysed depends on the task in hand. It can be low, e.g. 1,000 to 10,000 for strongly expressed genes. For the analysis of weakly expressed genes, it must be increased, e.g. to 100,000 or more.

If, for example, 100,000 individual gene products are analysed simultaneously, weakly expressed genes, e.g. approximately 100 mRNA molecules/cell (corresponding to approximately 0.02% of the total mRNA) are also represented in the reaction by on average 20 identified gene products.

The following method can be used as internal control of hybridisation, immobilisation and the sequencing reaction:

One or several nucleic acid chains with known sequences can be used as controls. The composition of these control sequences is not restricted, provided they do not interfere with the identification of the gene products. During the sequence analysis of the mRNA specimens, RNA control specimens are used, for the analysis of the cDNA specimens, DNA control specimens are used correspondingly. These specimens are preferably used simultaneously in all the steps. They can be added e.g. after mRNA isolation. In general, the control specimens are prepared for sequence analysis in the same way as the gene products to be analysed.

The control sequences are added to the gene products to be analysed in known, firmly set concentrations. The concentrations of the control specimens can vary; preferably, these concentrations are between 0.01% and 10% of the total concentration of the specimen to be analysed (100%). If the concentration of the mRNA is 10 ng/μl, for example, the concentration of control specimens is between 1 pg/μl and 1 ng/μl.

During the quantitative analysis of the gene expression, the general metabolic activity of the cells must also be taken into consideration, in particular if a comparison of the expression of certain genes is desired under different external conditions.

The change in the expression level of a certain gene can occur as a result of the change in the transcription rate of this gene or as a result of a global change in the gene expression in the cell. To observe the metabolic states in the cell, the expression of the so-called “housekeeping genes” can be analysed. For example in the case of a lack of important metabolites, the general expression level in the cell is low such that constitutively expressed genes also have a low expression level.

In principle, all constitutively expressed genes can serve as “housekeeping genes”. The transferrin receptor gene or the beta actin gene can be mentioned as examples.

The expression of these housekeeping genes consequently serves as a reference parameter for the analysis of the expression of other genes. The sequence determination and quantification of the expression of the housekeeping genes is preferably part of the analysis program for gene expression.

### 4.6 Example of Polymerase

When selecting the polymerase, the type of fixed nucleic acid (RNA or DNA) plays a decisive role.

If RNA is used as NACs or NACFs or gene product (e.g. mRNA) in the sequencing reaction, commercial RNA-dependent DNA polymerases can be used e.g. AMV Reverse Transcriptase (Sigma), M-MLV Reverse Transcriptase (Sigma), HIV reverse transcriptase without RNAse activity. All reverse transciptases must be largely free from RNAse activity (“Molecular cloning” 1989, Ed. Maniatis, Cold Spring Harbor Laboratory).

If DNA is used as NACs or NACFs or gene product (e.g. cDNA), all DNA-dependent DNA polymerases without 3′-5′ exonuclease activity are suitable, in principle, as polymerases (“DNA Replication” 1992 Ed. A. Kornberg, Freeman and company NY), e.g. modified T7 polymerase of the type “Sequenase Version 2” (Amersham Pharmacia Biotech), Klenow fragment of DNA polymerase I without 3′-5′ exonuclease activity (Amersham Pharmacia Biotech), polymerase beta of different origin (Animal Cell DNA Polymerases” 1983, Fry M., CRC Press Inc., commercially available from Chimerx), thermally stable polymerases such as Taq polymerase (GibcoBRL), proHA-DNA-polymerase (Eurogentec).

Polymerases with 3′-5′ exonuclease activity can be used (e.g. Klenow fragment of E. coli polymerase I) provided reaction conditions are chosen which suppress existing 3′ -5′ exonuclease activity such as e.g. a low pH (pH 6.5) in the case of the Klenow fragment (Lehman and Richardson, J. Biol. Chem. 1964 version 239 page 233) or the addition of NaF to the incorporation reaction. Another possibility consists of the use of NTs* with a phosphorothioate compound (Kunkel et al. PNAS 1981, version 78 page 6734). In this case, incorporated NTs* are not attacked by the 3′-5′ exonuclease activity of the polymerase. In the following, all of these types of polymerase will be referred to as “polymerase”.

### 4.7 Example of Modified Nucleotides

For highly parallel sequencing with individual molecules (parallel sequencing analysis of up to 10,000,000 nucleic acid molecules), it is important that each incorporated NT* is identified during the sequencing reaction. A precondition for this is that only a single NT* is incorporated into the nucleic acid chain per cycle.

This is achieved by reversible coupling of a group leading to termination. This group can be coupled both to the base (e.g. position 5 of the pyrimidines or position 7 of the 7-deazapurines) and to the 3′ position of ribose or 2′ deoxyribose of the nucleotide, respectively.

If this group is coupled to the base, it represents a sterically demanding group which, by its chemical structure, changes the properties of the NTs* coupled to this group in such a way that these cannot be incorporated in succession by a polymerase in an extension reaction. If a reaction mixture containing only modified NTs* is used in the reaction, the polymerase is capable of incorporating only a single NT*. The incorporation of a next NT* is sterically hindered. These NTs* consequently act as terminators of the synthesis. After removing the sterically demanding group, the next complementary NT* can be incorporated . Because these NTs* do not represent any absolute hindrance for the continued synthesis but only for the incorporation of a further labelled NT*, they are referred to as semi-terminators.

General Structure of the NT* with Steric Hindrance:

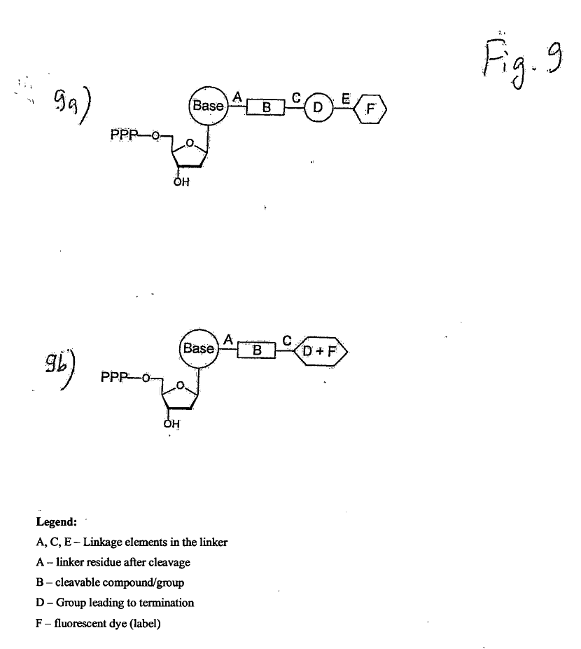

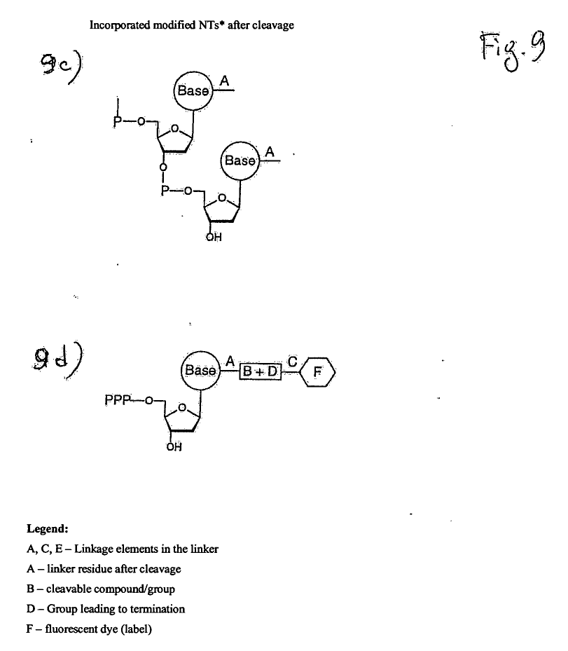

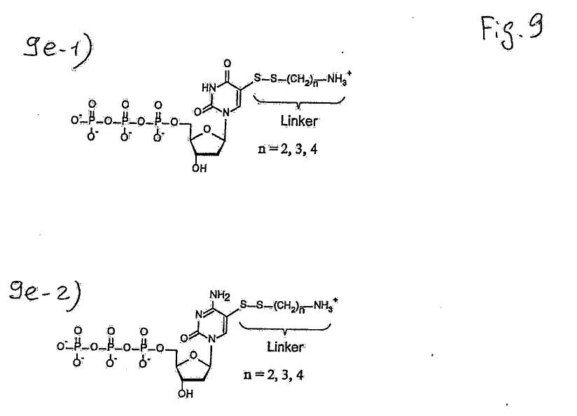

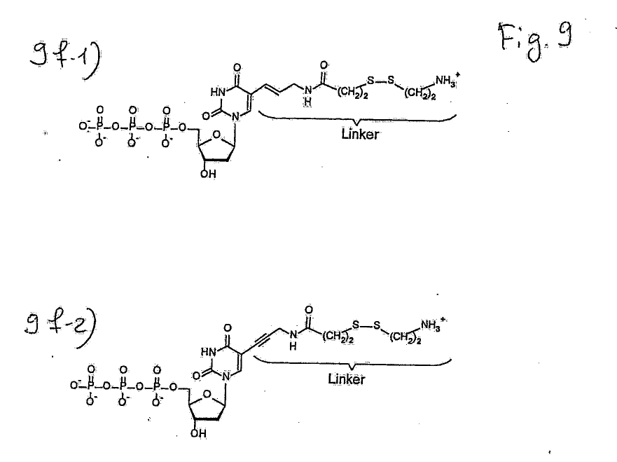

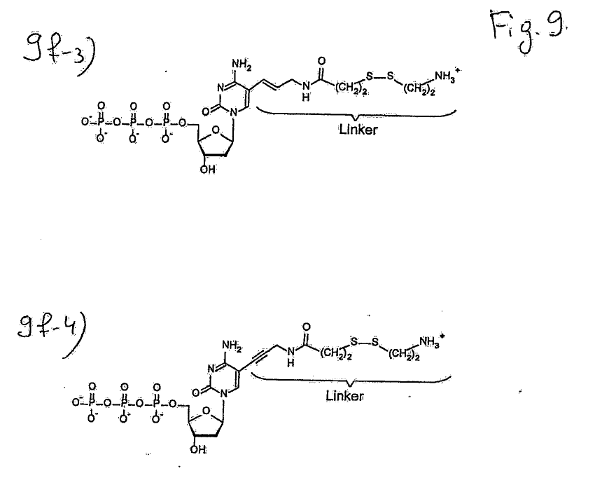

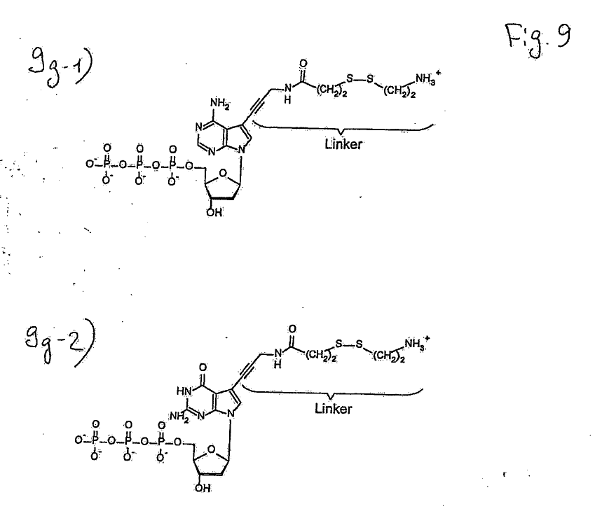

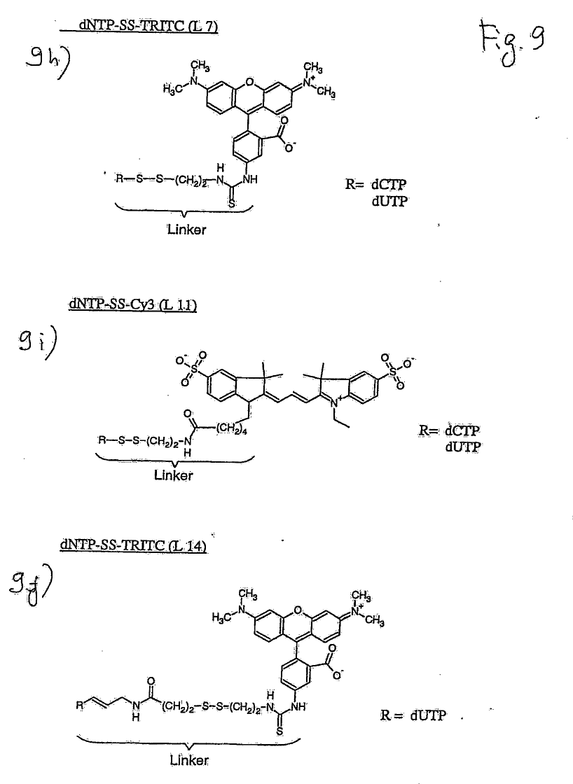

Their joint features are illustrated in FIG. 9a, b, d. This structure is characterised in that a steric group (D) and the fluorescent label (F) are bound to the base via a cleavable linker (A-E).

Deoxynucleoside triphosphates having adenosine (A), guanosine (G), cytidine (C) and uridine (U) as nucleoside residue serve as the basis for the NT*. Instead of guanosine, inosine can be used.

Nature of the sterically demanding group.

Group (D) (FIG. 9a, b, d) represents a hindrance for the incorporation of a further complementary labelled NT* by a polymerase.

Biotin, digoxigenin and fluorescent dyes such as fluorescein, tetramethyl rhodamine and Cy3 dye are examples of such a sterically demanding group (Zhu et al. Cytometry 1997, volume 28, page 206, Zhu et al. NAR 1994, volume 22, page 3418, Gebeyehu et al., NAR 1987, volume 15, page 4513, Wiemann et al. Analytical Biochemistry 1996, volume 234, page 166, Heer et al. BioTechniques 1994 volume 16 page 54). The chemical structure of this group is not restricted provided it does not interfere with the incorporation of the labelled NT* to which it is coupled and causes no irreversible interference with the enzymatic reaction.

This group can occur as an independent part in the linker (6a) or be identical to the dye (9b) or the cleavable group (9d). By cleaving the linker, this sterically demanding group (D) is removed after the detection of the signal such that the polymerase is capable of incorporating a further labelled NT*. In the case of a structure as in 6d, the steric group is removed by the cleavage.

In a preferred embodiment, the fluorescent dye takes on the function of such a sterically demanding group such that a labelled nucleotide exhibits a structure as depicted in FIG. 9b.

In another preferred embodiment, the photolabile cleavable group takes on the function of such a sterically demanding group (FIG. 9d).

Linker:

The label (fluorescent dye) is bound to the base preferably via a spacer of different length, a so-called linker. Examples of linkers are given in FIG. 9e, f, g, i, j. Examples of the coupling of a linker to the base can be found in the following sources (Hobbs et al. U.S. Pat. No. 5,047,519, Khan et al. U.S. Pat. No. 5,821,356, Klevan et al. U.S. Pat. No. 4,828,979, Hanna M. Method in Enzymology 1996 volume 274, page 403, Zhu et al. NAR 1994 volume 22 page 3418, Herman et al. Methods in Enzymology 1990 volume 184 page 584, J L Ruth et al. Molecular Pharmacology 1981 volume 20 page 415, L Ötvös et al. NAR 1987 volume 15 page 1763, G. E. Wright et al. Pharmac Ther. 1990 volume 47, page 447 “Nucluotide Analogs; Synthesis and Biological Function” K. H. Scheit 1980, Wiley-Interscience Publication, “Nucleic acid chemistry” Ed. L. B. Townsend, volume 1-4, Wiley-Interscience Publication, “Chemistry of Nucleosides and Nucleotides” Ed. L. B. Townsend, volume 1-3, Plenum Press).

The overall length of the linker can vary. It corresponds to the number of carbon atoms in sections A, C, E (Fig. a, b, d) and is preferably between 3 and 20. In the optimal case, it is between 4 and 10 atoms long. The chemical composition of the linker (sections A, C, E in FIG. 9a, b, d) is not restricted provided it remains stable under reaction conditions and does not interfere with the enzymatic reaction.

Cleavable Compound, Cleavage:

The linker carries a cleavable compound or cleavable group (section (B) in FIG. 9a, b, d). This cleavable compound permits the removal of the label and the steric hindrance at the end of each cycle. Its selection is not restricted provided it remains stable under the conditions of the enzymatic sequencing reaction, causes no irreversible interference with the polymerase and can be cleaved off under mild conditions. “Mild conditions” should be understood to be those conditions which do not destroy the gene product primer complex, the pH being preferably between 3 and 11, for example, the temperature between 0° C. and a temperature value (x). This temperature value (x) depends on the Tm of the gene product primer complex (Tm stands for “melting point”) and is calculated, for example, as Tm (gene product primer complex) minus 5° C. (if, for example, Tm is 47° C., the maximum temperature is 42° C.; under these conditions, ester compounds, thioester compounds, disulphide compounds and photolabile compounds, in particular, are suitable as cleavable compounds).

Preferably the above-mentioned groups belongs to compounds which are chemically or enzymatically cleavable or photolabile. Ester compounds, thioester compounds and disulphide compounds are preferred as examples of chemically cleavable groups (“Chemistry of protein conjugation and crosslinking” Shan S. Wong 1993 CRC Press Inc., Herman et al. Method in Enzymology 1990 volume 184 page 584, Lomant et al. J. Mol. Biol. 1976 volume 104 243, “Chemistry of carboxylic acid and esters” S. Patei 1969 Interscience Publ.). Examples of photolabile compounds can be found in the following literature references: “Protective groups in organic synthesis” 1991 John Willey & Sons, Inc., V. Pillai Synthesis 1980 page 1 V. Pillai Org. Photochem. 1987 volume 9 page 225, thesis “Neue photolabile Schutzgruppen für die lichtgesteuerte Oligonucleotidsynthese” (New photolabile protective groups for light-controlled oligonucleotide synthesis), H. Giegrich, 1996, Constance, thesis “Neue photolabile Schutzgruppen für die lichtgesteuerte Oligonucleotidsynthese” New photolabile protective groups for light-controlled oligonucleotide synthesis, S. M. Bühler, 1999 Constance).

The position of the cleavable compound/group in the linker is preferably not more than 10 atoms, even more preferably not more than 3 atoms away from the base. Particularly preferably, the cleavable compound or group is situated directly on the base.

The cleavage and removal step is present in every cycle and must take place under mild conditions (compare above) such that the nucleic acids are not damaged or modified.

Preferably, cleavage takes place chemically (e.g. in a mildly acidic or basic environment for an ester compound or by the addition of a reducing agent, e.g. dithiothreitol or mercaptoethanol (Sigma) during the cleavage of the disulphide compound) or physically (e.g. by exposing the surface to light at a certain wave length for the cleavage of a photolabile group, thesis “Neue photolabile Schutzgruppen für die lichtgesteuerte Oligonucleotidsynthese” (New photolabile protective groups for light-controlled oligonucleotide synthesis, H. Giegrich, 1996, Constance).

After the cleavage, a linker residue (A) remains on the base (FIG. 9c). If the mercapto group liberated on the linker residue after cleavage interferes with further reactions, it can be chemically modified by different known means (e.g. by disulphide or iodine acetate compounds).

Overall, the size, charge and chemical structure of the label, the length of the cleavable linker and the linker residue as well as the selection of the polymerase play an important part. They jointly determine whether the labelled NT* is incorporated by the polymerase into the growing nucleic acid chain and whether, as a result, the incorporation of the next labelled NT* is prevented. Two conditions need to be taken into consideration in this respect:

On the one hand, it is important that the polymerase is able to further extend the nucleic acid chain with the incorporated modified NT* after the cleavage of the linker. It is also important for the linker radical “A” (FIG. 9c) not to cause any major interference with continued synthesis after the cleavage. On the other hand, incorporated, non-cleaved NTs* must be a hindrance. Many NTs* suitable for the reaction can be synthesised. For each combination of polymerase and NTs*, a series of preliminary tests must be carried out individually during which the suitability of a certain type of NT* for sequencing is tested.

The buffer conditions are selected in line with the information provided by the manufacturer of the polymerase. For non-thermally stable polymerases, the reaction temperature is selected according to the information provided by the manufacturer (e.g. 37° C. for sequenase version 2); for thermally stable polymerases (e.g. Taq polymerase), the reaction temperature is maximum equal to the temperature value (x). This temperature value (x) depends on the Tm of the gene product primer complex and is calculated e.g. as Tm (gene product primer complex) minus 5° C. (if Tm is 47° C., for example, the maximum reaction temperature is 42° C.). In the following, these buffer conditions and this reaction temperature will be referred to as “optimum buffer and temperature conditions”.

The reaction period (corresponds to the period of the incorporation step in a cycle) is probably less than one hour long, ideally the reaction period is between 10 seconds and 10 minutes.

The following combinations deserve to be mentioned as examples of suitable combinations between NT* and polymerase:

If DNA (e.g. cDNA) is used in the reaction, NT* with a short linker residue can be used (FIG. 9e, h, i): dNTP-SS-TRITC (L7), dNTP-SS-Cy3 (L11) and/or NT* with a long linker residue (FIG. 9f, g, j): dNTP-SS-TRITC (L14) can be used in combination with sequenase version 2, Taq polymerase (GibcoBRL), ProHA-DNA-Polymerase (Eurogentec) or Klenow fragment of DNA polymerase I from E. coli without 3′-5′ exonuclease activity (Amersham Pharmacia Biotech).

If RNA (e.g. mRNA) is used in the reaction, NT* with a short linker residue can be used (FIG. 9e, h, I): dNTP-SS-TRITC (L7), dNTP-SS-Cy3 (L11) and/or NT* with a long linker residue (FIG. 9f, g, j): dNTP-SS-TRITC (L14) can be used in combination with AMV-reverse transcriptase (Sigma), M-MLV reverse transcriptase (Sigma), HIV reverse transcriptase without RNAse activity.

Syntheses:

Modified dUTP with a long cleavable linker (FIG. 9f-1). As starting substances, 5-(3 amino allyl)-2′-dexoyuridine-5′triphosphate, AA-dUTP (Sigma), 3,3′-dithio-bis(propionic acid-N-hydroxysuccinimide ester), DTBP-NHS, (Sigma), 2-mercaptoethylamine, MEA, (Sigma) can be used. To 100 μl of 50 mmole/l solution of AA-dUTP in 100 mmole/l borate buffer, pH 8.5, 3 equivalents of DTBP-NHS in DMF (25 μl 0.4 mole/l solution) are added. The reaction mixture is incubated at room temperature for 4 hours. Subsequently, concentrated ammonium acetate solution (pH 9) is added until the overall concentration of CH₃COONH₄ in the reaction solution is 100 mmole/l and the reaction mixture is incubated for a further hour. Subsequently, 200 μl of 1 mole/l of MEA solution, pH 9, are added to this mixture and incubated at room temperature for 1 hour. Subsequently, a saturated solution of I₂ in 0.3M K1 solution is added dropwise to this mixture until the iodine colour remains in the solution. The modified nucleotides are separated off from other reaction products in a DEAE cellulose column in ammonium carbonate gradient (pH 8.5). The isolation of the nucleotide with the cleavable linker takes place on RP-HPLC. Dyes can then be coupled to this linker by different methods (“Handbook of Fluorescent Probes and Research Chemicals” 6^{th} ed. 1996, R Haugland, Molecular Probes, Waggoner Method in Enzymology 1995 volume 246, page 362, Jameson et al. Method in Enzymology 1997, volume 278, page 363).

Other nucleotide analogues (e.g. according to Hobbs et al, U.S. Pat. No. 5,047,519, Khan et al. U.S. Pat. No. 5,821,356) can be used in the reaction such that nucleotide analogues with the structures shown in FIGS. 9f-2, 3, 4 and 9g-1, 2 can be produced.

Coupling of TRITC (Tetramethyl rhodamine-5-isothiocyanate, Molecular Probes) is given as an example of coupling of a dye to a linker (NT* structure FIG. 9j):

The dNTP (300 nmole) modified with the cleavable linker is dissolved in 30 μl of 100 mmole/l sodium borate buffer, pH 9 (10 mmole/l NT*). 10 μl of 10 mmole/l of TRITC in DMF are added and incubated for four hours at room temperature. The purification of the NT* modified with the dye takes places via RP-HPLC in methanol-water gradient. In a similar way, other dyes can be coupled to the amino group of the linker.

The NT* produced in this way satisfies the requirements of incorporation into the DNA strand, of fluorescence detection and chain termination following incorporation and the elimination of the hindrance necessary for the success of the process.

Example of the cleavage of disulphide compound in modified NT*. The cleavage takes place by the addition of 20 to 50 mmole/l of DTT or mercaptoethanol (Sigma) solution, pH 8, onto the reaction surface. The surface is incubated with this solution for 10 minutes, the solution is then removed and the surface washed with a buffer solution to remove residues of DTT and/or mercaptoethanol.

Modified dUTP (dUTP-SS—CH₂CH₂NH₂) with a short cleavable linker (FIG. 9e-1). The following serve as starting substances: Bi-dUTP, synthesised according to Hanna (Method in Enzymology 1989, volume 180, page 383), 2-mercaptoethylamine, MEA, (Sigma).

To 400 μl of 100 mmole/l bis-dUTP in 40 mmole/l of borate buffer, pH 8.5, 100 μl of 100 mmole/l MEA solution, pH 8.5, in H₂O are added and incubated for 1 hour at room temperature.

Subsequently, a saturated solution of I2 in 0.3 mmole/l of K1 solution is added dropwise to this mixture until the iodine colour remains in the solution. The nucleotides (bis-dUTP and dUTP-SS—CH₂CH₂NH₂) can be separated off from other reaction products e.g. by an ethanol precipitation or on a DEAE cellulose column in ammonium carbonate gradient (pH 8.5). Bis-dUTP does not interfere with the subsequent coupling of a dye to the amino group of the linker so that the separation of the dUTP-SS—CH₂CH₂NH₂ from bis-dUTP can take place in the final purification step.

dCTP (FIG. 9-e2) can be modified in a similar way, bis-dCTP serving as starting substance (synthesised according to Hanna et al. Nucleic Acid Research 1993, volume 21, page 2073).

Further NT* (dUTP* and dCTP*) with a short linker residue can be synthesised in a similar way, wherein NT*, for example, may have the following structures (FIG. 9e):
dUTP-SS—(CH₂)ₙ—NH₂, FIG. 9e-1.
dCTP-SS—(CH₂)ₙ—NH₂, FIG. 9e-2.
wherein n is between 2 and 6, preferably between 2 and 4, further examples are:
dUTP-SS—(CH₂)ₙ—X—CO—(CH₂)ₘ-Z
dUTP-SS—(CH₂)ₙ—X—CO—Y—(CH₂)ₘ-Z
dCTP-SS—(CH₂)ₙ—X—CO—(CH₂)ₘ-Z
dCTP-SS—(CH₂)ₙ—X—CO—Y—(CH₂)ₘ-Z
X═NH, O, S
Y═NH, O, S
Z═NH₂, OH, dye
wherein (n+m) is between 4 and 10, preferably between 4 and 6.

It is then possible to couple dyes to the linker by using different methods (“Handbook of Fluorescent Probes and Research Chemicals” 6^{th} ed. 1996, R Haugland, Molecular Probes, Waggoner Method in Enzymology 1995 volume 246, page 362, Jameson et al. Method in Enzymology 1997, volume 278, page 363).

As an example of the coupling of a dye to the linker, the coupling of the FluoroLink™ Cy3 monofunctional dye (Amersham Pharmacia Biotech) (NT*-structure FIG. 9i) is indicated. This is a monofunctional NHS ester fluorescent dye. The reaction is carried out in line with the manufacturer's information:

The dNTP (300 nmole) modified with the cleavable linker is dissolved in 300 μl of 100 mmole/l of sodium borate buffer, pH 8.5. Dye (300 nmole) is added and incubated for 1 h at room temperature. The purification of the NT* modified with the dye takes place via RP-HPLC in a methanol-water gradient.

As a further example of coupling of a dye to the linker, coupling of TRITC (tetramethyl rhodamine-5-isothiocyanate, Molecular Probes) is indicated (dUTP-SS-TRITC, FIG. 9h).

The dNTP (300 nmole) modified with the cleavable linker is dissolved in 30 μl of 100 mmole/l sodium borate buffer, pH 9 (10 mmole/l NT*). For this purpose, 10 μl of 10 mmole/l TRITC are introduced into DMF and incubated for 4 h at room temperature. The purification of the NT* modified with the dye takes places via RP-HPLC in methanol-water gradient.

The NT* produced in this way satisfies the requirements of incorporation into the DNA strand, of fluorescence detection and chain termination following incorporation and the elimination of the hindrance necessary for the success of the process.

Examples of the cleavage of the disulphide compound in modified NT*. The cleavage takes place by the addition of 20 to 50 mmole/l of dithiothreitol solution (DTT) or mercaptoethanol solution (Sigma), pH 8, onto the reaction surface. The surface is incubated with this solution for 10 minutes, the solution is then removed and the surface washed with a buffer solution to remove residues of DTT and/or mercaptoethanol.

General NT Structure with a Group Coupled to Ribose and/or 2′-deoxyribose and Leading to Termination

In the processes, different NT*s can be used (preferably 2′-deoxynucleotide triphosphates) which carry a substituent at their 3′ position of the ribose ring (the group leading to termination). This substituent can lead to the termination of the incorporation reaction either alone or together with the fluorescent dye and be cleaved off from the nucleotide under mild conditions. A fluorescent dye characteristic of the NT* concerned is coupled to this substituent such that the substituent also takes on the role of a linker between the nucleotide and the fluorescent dye. Preferably, the fluorescent dye is coupled to this linker by a bond cleavable under mild conditions.

“Mild conditions” should be understood to mean cleavage conditions leading to neither denaturing of the primer nucleic acid complex nor to the cleavage of its individual components.

Formulae (1-3) represent examples of the reversible cleavable terminators:
NT-3′-O—S (1)-F 1)
NT-3′-O—S (2)-N—F 2)
NT-3′-O—S (2)-N-L-F 3)

NT-3′-O—represents the 2′-deoxynucleotide triphosphate residue.

S(1)—represents a substituent (formula 1) which can be cleaved off from NT* under mild conditions. A fluorescent dye (F) is coupled to this substituent.

S(2)-N—represents a further substituent (formula 2 and 3) which can be cleaved off from NT* under mild conditions. This substituent is linked with the fluorescent dye (F) by a group (N) cleavable under mild conditions. The fluorescent dye can be coupled directly to the cleavable group (formula 2) or by a further linker (L) (formula 3).

Examples of NT* structures, NT* synthesis, regarding the polymerase selection for the incorporation reaction, reaction conditions of the NT* incorporation reaction and cleavage reaction are described in (Kwiatkoxski WO Patent 01/25247, Kwiatkowski U.S. Pat. No. 6,255,475, Conard et al. U.S. Pat. No. 6,001,566, Dower (U.S. Pat. No. 5,547,839), Canard et al (U.S. Pat. No. 5,798,210), Rasolonjatovo (Nucleosides & Nucleotides 1999, volume 18 page 1021), Metzker et al (NAR 1994, volume 22 page 4259), Welch et al. (Nucleosides & Nucleotides 1999 volume 18 page 197).

Cleavable Bond Between the Nucleotide and the Substituent, Cleavage:

The substituent leading to termination is coupled to the NT by a bond cleavable under mild conditions.

Examples of these compounds are esters and acetals.

Preferably, the cleavage of the ester takes place within the basic pH range (e.g. 9 to 11). The cleavage of acetals takes place in the acidic range (e.g. between 3 and 4).

Esters can be cleaved off also enzymatically by polymerases or esterases.

According to a preferred embodiment of the invention, the substituent is cleaved off together with the fluorescent dye in one step.

Cleavable Bond Between the Substituent and the Fluorescent Dye, Cleavage:

According to a further preferred embodiment of the invention, the fluorescent dye is coupled to the substituent by a group cleavable under mild conditions.

Preferably the above-mentioned group belongs to compounds which are chemically or enzymatically cleavable or photolabile.

Ester compounds, thioester compounds, disulphide compounds and photolabile compounds are particularly suitable for use as cleavable compound between the substituent and the fluorescent dye.

Ester compounds, thioester compounds and disulphide compounds are preferred as examples of chemically cleavable groups (“Chemistry of protein conjugation and crosslinking” Shan S. Wong 1993 CRC Press Inc., Herman et al. Method in Enzymology 1990 volume 184 page 584, Lomant et al. J. Mol. Biol. 1976 volume 104 243, “Chemistry of carboxylic acid and esters” S. Patei 1969 Interscience Publ.). Examples of photolabile compounds can be found in the following literature references: “Protective groups in organic synthesis” 1991 John Willey & Sons, Inc., V. Pillai Synthesis 1980 page 1 V. Pillai Org. Photochem. 1987 volume 9 page 225, thesis “Neue photolabile Schutzgruppen für die lichtgesteuerte Oligonucleotidsynthese” (New photolabile protective groups for light-controlled oligonucleotide synthesis), H. Giegrich, 1996, Constance, thesis “Neue photolabile Schutzgruppen für die lichtgesteuerte Oligonucleotidsynthese” (New photolabile protective groups for light-controlled oligonucleotide synthesis, S. M. Bühler, 1999 Constance).

The cleavage step is present in every cycle and must take place under mild conditions such that the nucleic acids are not damaged or modified.

Preferably, cleavage takes place chemically (e.g. in a mildly acidic or basic environment for an ester compound or by the addition of a reducing agent, e.g. dithiothreitol or mercaptoethanol (Sigma) during the cleavage of the disulphide compound) or physically (e.g. by exposing the surface to light at a certain wave length for the cleavage of a photolabile group, thesis “Neue photolabile Schutzgruppen für die lichtgesteuerte Oligonucleotidsynthese” (New photolabile protective groups for light-controlled oligonucleotide synthesis, H. Giegrich, 1996, Constance).

In this embodiment, the fluorescent dye is cleaved off first following the detection and only then the substituent which is coupled to the 3′ position and leads to termination.

The invention is to be further illustrated by way of a few diagrammatic figures.

Legends to Figures:

FIG. 1 Diagrammatic representation of an embodiment of the automated sequencing device.

101 Source of light for the epifluorescence mode

102 Focusing optics (1)

103 Shutter (S1)

104 Beam of light of the excitation light

105 Set of filters or several sets of filters for the selection of the light wavelength and colour separator

106 Lens system

107 Reaction platform with

107a Pump

107b Storage vessel

107c Valves

108 Translation table (scanning table)

109 Condenser

110 Mirror

111 Shutter (S2)

112 Focusing optics (2)

113 Source of light for transmission mode

114 Beam of light of the transmission light

115 Tube optics 1

116 Detection device

The housing is not shown.

Flow chart with an example of the course of essential operating steps:

During initialisation, the user selects the parameters for the sequencing reaction. The wing parameters are set:
1) The type of investigation, e.g. sequencing of long NACs or gene expression analysis
2) The average length of the immobilised NACs and/or NACFs
3) The average number of NT*s incorporated per NAC
4) The sensitivity and specificity of the analysis

In the section precyclic reactions, NACs and/or NACFs are fixed in MFC in the form of NAC primer complexes and/or NACF primer complexes. The aim of this section is to immobilise the samples to be investigated in an optimum density (compare example Immobilisation). The parameters of the hybridisation step (primer and PBS composition, composition of the solution, optimum hybridisation and wash temperature, primer immobilisation density on the surface, concentration of the NACs) are preferably known and, together with the duration of the hybridisation step, determine the immobilisation density of the NACs.

In the section cyclic reactions, labelled NT*s are incorporated into the complementary strand of immobilised NACs and/or NACFs and the signals of incorporated NT*s are detected by scanning the reaction surface, identified and allocated to specific types of NT* (signal processing).

In the section Data processing, the construction of the sequences is effected from individual identified and allocated NT*s.

FIG. 3 represents a “state of the art” epifluorescence microscope which can be integrated into the automated sequencing device.

117 Conduction optics to the lens system

118 Tube optics 2

119 Ocular optics

The housing is not shown

FIG. 4a An advantageous embodiment of the detection device.

It is characterised in that
1) several sets of filters are fitted in a filter revolver or filter slide 120,
2) the scanning table 108, the filter revolver or the filter slide 120, the shutter 103 and the thermostat unit 111 are connected with the pump and the control valves (not shown in this Fig.) for the control of the operating step by means of the computer 121.

For focusing and adjustment, the transmission light is used in this embodiment.

FIG. 4b This exemplary embodiment is characterised by the following features:

The automated sequencing device is equipped with a device (122) for controlling and regulating the intensity of the excitation light. This intensity control can be effected by changing the output of the source of light (101), for example. The device represents part of the control circuit for the light intensity and is connected to the central computer unit.

2) For focusing and for the adjustment images, the fluorescence signal of the pattern connected to the reaction surface is used (compare Example of Detection).

FIG. 5 An example of the detection device, characterised in that one or several lasers (in this example 2, laser 123 and laser 124) are used as sources of light. These lasers can be integrated into the housing of the sequencing device or connected with the automated sequencing device by fibre optics. For the modulation of the excitation light with respect to time (the exposure time is preferably between 0.1 msec and 1 sec), a special device 125, for example, is used.

FIG. 6 Examples of the reaction platform:

FIG. 6a Overall view of the reaction platform. The following is represented: an embodiment with four differently labelled NT*s which are used simultaneously in the incorporation reaction.

201 Fixing plate

202 Feed connection

203 Discharge connection

204a Chip with MFC

204b MFC

205 Discharge hose

206 Valve for pump

207 Pump

208a, b, c, d Feed hoses for reaction solution NT*(n)

209 Feed hose for wash solution

210 Feed hose for sample solution

211 Feed hose for cleavage solution

212a, b, c, d Valves for reaction solution NT*(n)

213 Valve for wash solution

214 Valve for sample solution

215 Valve for cleavage solution

216a, b, c, d Storage vessel for reaction solution NT*(n)

217 Storage vessel for wash solution

218 Storage vessel for sample solution

219 Storage vessel for cleavage solution

220 Thermostat unit

221 Aperture for transmission light

222 Cover plate

223 Base plate

224 Sensor

FIG. 6b Overall representation of the chip with the microfluid channel (MFC). The channel may contain expanded and split areas leading to an enlargement of the reaction surface. The choice of the form of the MFC depends on the number of object fields which need to be scanned: in the case of a large number, MFC with a relatively large reaction surface will be used.

FIG. 6c Overall representation of the distribution device. An embodiment is illustrated with the four differently labelled NT*s which are used simultaneously in the incorporation reaction.

FIG. 6d Overview representation of the distribution device. An embodiment with the four labelled NT*s is illustrated, only two differently labelled NT*s being simultaneously used in the incorporation reaction in cycle N. The other two are used in cycle N+1.

FIG. 6e Overview representation of the distribution device. An embodiment is illustrated in the case of which only one NT* is used per cycle, all four NT*s having the same label.

FIG. 6f Overview representation of the reaction platform. An embodiment is illustrated in the case of which a sensor 224 is capable of controlling the replacement of the solutions e.g. by optical means.

FIG. 7 Diagrammatic overview representation of the scanning process of the reaction surface in one cycle. For this purpose, 2D images (301) are taken of several object fields (302). Fluorescence signals (303) of individual incorporated NT*s have characteristic co-ordinates (X(n), Y(n).

FIG. 8 Detections step in an embodiment with 4NT*s (NT*_{1,2,3,4}) labelled with different dyes. After setting the X, Y co-ordinates in an object field, the focus position of the reaction surface is verified and/or adjusted. The verification takes place e.g. in the transmission light mode, shutter S2 (111) being open, shutter S1 (103) closed. Subsequently, the fluorescence signals are recorded. In this example, a specific set of filters (NT*(n)) is used for each dye. During the exposure time, shutter S1 (103) is open and shutter S2 (111) closed.

FIG. 9 Examples of nucleotide structures used in the process.

1. Automated sequencing device for parallel sequencing of a population of individual nucleic acid chain molecules fixed on a plane surface, this sequencing occurring by the sequential construction of a strand complementary to the fixed nucleic acid chain concerned with the nucleotides reversibly labelled with fluorescent dyes, the sequential construction taking place in cyclic reactions. This automated sequencing device comprises the following elements:
An optical system for the detection of signals of individual molecules, which system comprises the following components:
A source of electromagnetic radiation for the excitation of the fluorescence of dyes which are coupled to the modified nucleotides,
A device for focusing the electromagnetic radiation used for the excitation of fluorescence and for collecting emitted electromagnetic radiation (fluorescence signals) of individual dye molecules which are coupled to the modified nucleotide molecules incorporated into the strands complementary to the nucleic acid chains to be sequenced,
A filter device for selecting wavelengths of the electromagnetic radiation used for the excitation of the fluorescence and of electromagnetic radiation collected (fluorescence signals),
A detection device for the detection of the electromagnetic radiation, selected by the filter device (fluorescence signals), of individual dye molecules which are coupled to the modified nucleotide molecules incorporated into the strands complementary to the nucleic acid chains to be sequenced,
A translation device for the translation of the reaction platforms during scanning of the surface and for changing over between reaction platforms during the cycle steps,
One or several reaction platforms on the translation device for the execution of sequential reaction cycles with immobilised nucleic acid chains, these platforms permitting a simultaneous detection of the signals of many individual dye molecules which are coupled to modified nucleotide molecules incorporated into the strands complementary to the nucleic acid chains to be sequenced,
A housing for retaining the optical system, detection device and translation device,
An analytical device for the determination of sequences of fixed nucleic acid chains by way of signals, detected by the detection device, of individual modified nucleotides molecules to be incorporated into strands complementary to the nucleic acid chains to be sequenced,
A control device for controlling
a) the cycles in the reaction platform
b) the optical system
c) the translation device
d) the analysis device

2. Automated sequencing device according to claim 1 characterised in that the electromagnetic radiation used for the excitation of fluorescence is passed onto the reaction surface in the epifluorescence mode.

3. Automated sequencing device according to claim 1 characterised in that the optical system, the translation device and the housing are part of a fluorescence microscope.

4. Automated sequencing device according to claim 1 to 3 characterised in that the source of electromagnetic radiation is a lamp.

5. Automated sequencing device according to claim 4 characterised in that the source of the electromagnetic radiation is a mercury vapour lamp.

6. Automated sequencing device according to claim 1 to 3 characterised in that the source of electromagnetic radiation is one or several lasers.

7. Automated sequencing device according to claim 1 characterised in that the nucleic acid chains are fixed on a plane surface in the form of nucleic acid chain primer complexes.

8. Automated sequencing device according to claim 1 characterised in that several fluorescence signals of individual NT*s incorporated into different NACs and/or NACFs are detected simultaneously.

9. Automated sequencing device according to claim 1 characterised in that several nucleic acid chains are sequenced simultaneously.

10. Automated sequencing device according to claim 1 characterised in that it carries out the following process for the parallel sequence analysis of nucleic acid sequences (nucleic acid chains, NACs or their fragments, NACFs) whereby a cyclic build-up reaction of the complementary strand of the NACs and/or NACFs is carried out using one or several primers and one or several polymerases by
a) adding, to the NAC primer complexes or NACF primer complexes bound to the surface, a solution containing one or several polymerases and one to four modified nucleotides (NTs*) which are labelled with fluorescent dyes, the fluorescent dyes present on the NTs* in the case of the simultaneous use of at least two NTs* being selected such that it is possible to distinguish between the NTs* used by measuring different fluorescence signals, the NTs* being structurally modified such that the polymerase, following the incorporation of such an NT* into a growing complementary strand, is not capable of incorporating a further NT* into the same strand,
b) incubating the stationary phase obtained in stage a) under conditions suitable for extending the complementary strands, the complementary strands being extended in each case by one NT*,
c) washing the stationary phase obtained in stage b) under conditions suitable for removing NTs* not incorporated into a complementary strand,
d) detecting the individual NTs* incorporated into complementary strands by measuring the characteristic signal of the fluorescent dye concerned, the relative position of the individual fluorescence signals on the reaction surface being simultaneously determined,
e) cleaving off the fluorescent dyes and the group leading to termination from the NTs* added into the complementary strand in order to produce non-labelled (NTs or) NACs or NACFs,
f) washing the stationary phase obtained in stage e) under conditions suitable for removing the fluorescent dyes and the group,
stages a) to f) being repeated several times, if necessary,
whereby the relative position of individual NAC primer complexes or NACF primer complexes on the reaction surface and the sequence of these NACs or NACFs are determined by specific allocation, to the NTs, of the fluorescence signals detected in stage d) in successive cycles at the positions concerned.

11. Automated sequencing device according to claim 1 characterised in that it carries out the following process for the parallel sequence analysis of nucleic acid sequences (nucleic acid chains, NACFs) whereby
fragments (NACFs) of single-strand NACs with a length of approx. 50 to 1000 nucleotides are produced which may represent overlapping partial sequences of a total sequence,
the NACFs are bound in a random arrangement on a reaction surface by using a uniform primer or several different primers in the form of NACF primer complexes,
a cyclic build-up reaction of the complementary strand of the NACFs is carried out using one or several polymerases by
a) adding, to the NACF primer complexes bound to the surface, a solution containing one or several polymerases and one to four modified nucleotides (NTs*) which are labelled with fluorescent dyes, the fluorescent dyes present on the NTs* in the case of the simultaneous use of at least two NTs* being selected such that it is possible to distinguish between the NTs* used by measuring different fluorescence signals, the NTs* being modified structurally such that the polymerase, following the incorporation of such an NT* into a growing complementary strand, is not capable of incorporating a further NT* into the same strand,
b) incubating the stationary phase obtained in stage a) under conditions suitable for extending the complementary strands, the complementary strands being extended in each case by one NT*,
c) washing the stationary phase obtained in stage b) under conditions suitable for removing NTs* not incorporated into a complementary strand,
d) detecting the individual NTs* incorporated into complementary strands by measuring the characteristic signal of the fluorescent dye concerned, the relative position of the individual fluorescence signals on the reaction surface being simultaneously determined,
e) cleaving off the fluorescent dyes and the group leading to termination from the NTs* added into the complementary strand in order to produce non-labelled (NTs or) NACFs,
f) washing the stationary phase obtained in stage e) under conditions suitable for removing the fluorescent dyes and the group,
stages a) to f) being repeated several times, if necessary,
whereby the relative position of individual NACF primer complexes on the reaction surface and the sequence of these NACFs are determined by the specific allocation, to the NTs, of the fluorescence signals detected in stage d) in successive cycles at the positions concerned.

12. Automated sequencing device according to claim 1 characterised in that it carries out the following process for the highly parallel analysis of gene expression, whereby
single-strand gene products are provided,
the gene products are bound in a random arrangement on a reaction surface by using a uniform primer or several different primers in the form of gene product primer complexes,
a cyclic build-up reaction of the complementary strand of the gene product is carried out using one or several polymerases by
a) adding, to the gene product primer complexes bound to the surface, a solution containing one or several polymerases and one to four modified nucleotides (NTs*) which are labelled with fluorescent dyes, the fluorescent dyes present on the NTs* in the case of the simultaneous use of at least two NTs* being selected such that it is possible to distinguish between the NTs* used by measuring different fluorescence signals, the NTs* being modified structurally such that the polymerase, following the incorporation of such an NT* into a growing complementary strand, is not capable of incorporating a further NT* into the same strand,
b) incubating the stationary phase obtained in stage a) under conditions suitable for extending the complementary strands, the complementary strands being extended in each case by one NT*,
c) washing the stationary phase obtained in stage b) under conditions suitable for removing NTs* not incorporated into a complementary strand,
d) the individual NTs* incorporated into complementary strands by measuring the characteristic signal of the fluorescent dye concerned, the relative position of the individual fluorescence signals on the reaction surface being simultaneously determined,
e) cleaving off the fluorescent dyes and the group leading to termination from the NTs* added into the complementary strand in order to produce non-labelled (NTs or) gene products,
f) washing the stationary phase obtained in stage e) under conditions suitable for removing the fluorescent dyes and the group,
stages a) to f) being repeated several times, if necessary,
whereby the relative position of individual gene product primer complexes on the reaction surface and the sequence of these gene products are determined by the specific allocation, to the NTs, of the fluorescence signals detected in stage d) in successive cycles at the positions concerned and the identity of the gene products is determined from the partial sequences determined.

13. Reaction platform according to claim 1 for carrying out reaction steps, which platform comprises the following elements:
a replaceable chip with one or several microfluid channels
a distribution device for controlling the replacement of the solution in the chip
a thermostat unit for controlling the temperature in the chip.

14. Automated sequencing device according to claim 1 to 3 characterised in that the source of electromagnetic radiation is one or several laser diodes.


SEQUENCE LISTING (1 sequence)

SEQ ID NO: 1 - DNA, 52 bp, Artificial
  Description of Artificial sequence Exemplary nucleotide
cgtccgtatgatggtcattccatggtacgttagctcctagtaaaatcgtacc